(19) United States
(12) Patent Application Publication
(10) Pub. No.: US 2013/0031644 A1
(43) Pub. Date: Jan. 31, 2013

(54) AUTONOMOUS LUX REPORTER SYSTEM AND METHODS OF USE

(71) Applicants: Steven A. Ripp, Knoxville, TN (US); Gary S. Sayler, Blaine, TN (US); Daniel M. Close, Knoxville, TN (US); Michelle Connolly, Knoxville, TN (US); Theodore B. Henry, Plymouth (GB)

(72) Inventors: Steven A. Ripp, Knoxville, TN (US); Gary S. Sayler, Blaine, TN (US); Daniel M. Close, Knoxville, TN (US); Michelle Connolly, Knoxville, TN (US); Theodore B. Henry, Plymouth (GB)

(73) Assignee: University of Tennessee Research Foundation

(21) Appl. No.: 13/479,157

(22) Filed: May 23, 2012

Related U.S. Application Data
(60) Provisional application No. 61/489,170, filed on May 23, 2011.

(51) Int. Cl. G01N 21/76; A01K 67/027; C12N 5/10

(52) U.S. Cl. 800/3; 435/325; 800/20

(57) ABSTRACT

Disclosed are systems for expression of an autonomous lux reporter system in a vertebrate cell, such as mammalian or fish cell. In some examples the lux reporter system is operably connected to a pollutant-inducible DNA response element. Also disclosed are transgenic zebrafish, carrying pollution-inducible response elements, and methods of using such zebrafish to monitor pollutants.

### CROSS REFERENCE TO RELATED APPLICATIONS

This application claims the benefit of U.S. Provisional Application No. 61/489,170, filed May 23, 2011, which is specifically incorporated by reference herein in its entirety.

### STATEMENT OF GOVERNMENT INTEREST

This invention was made with government support under contract numbers CBET-0853780, awarded by the National Science Foundation and R21 CA127745-02 awarded by the National Institutes of Health. The government has certain rights in the invention.

### FIELD OF THE DISCLOSURE

This disclosure relates to the field of biosensors, and more specifically biosensors that incorporate bacterial luciferase and the use of such sensors.

### BACKGROUND

Exposure to numerous man-made and natural environmental agents poses a significant threat to human health. In order to protect human health, regulatory agencies have set limits on the concentrations levels and kinds of pollutants allowed to enter the environment, such as bodies of water. These environmental quality criteria are established on the basis of correlations between the concentration of a pollutant and risk assessment methodologies in humans.

In monitoring the quality of the aquatic environment, a major approach involves the quantitation of water, sediment, or tissue residue levels by analytical chemical methods, which are generally expensive, labor-intensive, and slow. This process usually includes the acquisition of a sample in the field, transport back to the analytical facility, sample processing, data collection, and, finally, data analysis. This is the more straightforward of the methods used-but also the more expensive, requiring extensive technical expertise in the analysis of pesticide, inorganic, non-pesticide organic, physical, and radiological parameters.

Biosensors, with their small size, relative simplicity, rapidity of operation, and continuous, real-time to near real-time monitoring capabilities, possess unique characteristics conducive to the high throughput and remote-based monitoring needs relevant to agricultural, environmental, pharmacological, and clinical sensing. While the most popular biosensors have traditionally incorporated enzymes or antibodies as their biorecognition elements, the development and use of whole-cell biosensors (those housed entirely within a bacterial or eukaryotic cell) has increased greatly in recent history because they possess some interesting advantages over their enzymatic and immuno-dependent counterparts. Primary among these advantages is the indication of bioavailability—the effect and interactions the analyte has on a living system. As opposed to analytical instruments that measure only the total concentration of a target analyte in a sample, whole-cell biosensors that measure bioavailability indicate that the analyte can be assimilated by or directly affect a living organism, thereby exposing possible toxic interactions. Another advantage of whole-cell bioreporters is that they can be designed to produce signal in a constitutive manner, regardless of the presence of a target analyte. This makes them valuable tools for localization and monitoring of cellular activities under in vivo, in vitro, or in situ conditions in ways not possible with standard analytical equipment.

### SUMMARY OF THE DISCLOSURE

Disclosed are vertebrate cells, such as mammalian or fish cells (for example zebrafish) that include one or more heterologous autonomous reporter vectors, wherein at least of the one or more heterologous autonomous reporter vectors comprises a nucleic acid sequence that encodes a bacterial luxA and a nucleic acid sequence that encodes a bacterial luxB. In some examples the luxA and the bacterial luxB are from Photorhabdus luminescens. In some examples, the luxA and luxB are codon optimized for expression in the vertebrate cell.

In some embodiments, the vertebrate cell further includes a nucleic acid sequence that encodes a bacterial luxC, a nucleic acid sequence that encodes a bacterial luxD, and a nucleic acid sequence that encodes a bacterial luxE. In some examples, the bacterial luxC, luxD, and luxE are from Photorhabdus luminescens. In some examples, the luxC, luxD, and luxE are codon optimized for expression in the vertebrate cell. In some embodiments, the vertebrate cell further includes a nucleic acid sequence that encodes a bacterial frp nucleotide sequence. In some examples, the bacterial frp is from V. harveyi. In some examples, the frp is codon optimized for expression in the vertebrate cell. The disclosed cells can be used as autonomous biosensors to detect for example toxic chemicals.

Also disclosed are transgenic zebrafish that include the aforementioned zebrafish cells. In some examples, the transgenic zebrafish is a biosensor that detects toxic chemicals. Methods of measuring contaminants in water using the disclosed zebrafish are also disclosed.

These and other features and advantages of the present disclosure will become apparent after a review of the following detailed description of the disclosed embodiments and the appended claims.

### BRIEF DESCRIPTION OF THE FIGURES

FIGS. 1A and 1B depict a schematic of the bioluminescent reaction catalyzed by the bacterial luciferase genes. FIG. 1A, the luciferase is formed from a heterodimer of the luxA and luxB gene products. The aliphatic aldehyde is supplied and regenerated by the products of the luxC, luxD, and luxE genes. The required oxygen and reduced riboflavin phosphate substrates are scavenged from endogenous metabolic processes, however, the flavin reductase gene (frp) aids in reduced flavin turnover rates in some species. FIG. 1B, the production of light, catalyzed by the products of the luxA and luxB genes, results from the decay of a high energy intermediate (R1=C₁₃H₂₇).

FIGS. 2A-2C depict schematics showing construction and expression of the full lux cassette using a two-plasmid system. The two-plasmid system takes advantage of IRES-based bicistronic expression to drive transcription/translation of all the genes required for autonomous bioluminescent production. FIG. 2A, the pLux_{AB} plasmid contains the genes responsible for production of the luciferase protein. Individual luxA and luxB genes were removed from their respective vectors and ligated into the pIRES vector using the unique NheI (N) and EcoRI (E) or SalI (S) and NotI (Nt) restriction sites. FIG. 2B, the pLux_{CDEfrp} plasmid expresses the genes required for production and regeneration of the aldehyde and FMNH₂ substrates. The individual luxE and luxC genes were cloned into a pIRES vector using the unique NotI (Nt) and SalI (S) or NheI (N) and EcoRI (E) restriction sites. FIG. 2C, a second pIRES vector was created that contained the luxD and frp genes inserted at the same sites. The entire luxC-IRES-luxE fragment was then inserted under the control of the EF1-α promoter in pBudCE4.1 using the unique XhoI (X) and SfiI (St) restriction sites, while the luxD-IRES-frp fragment was inserted under the control of the CMV promoter using the unique PsiI (P) and BamHI (B) restriction sites.

FIG. 3 is a graph showing growth rates of lux-containing HEK293 cells. Growth curve analysis of cells containing no plasmids (negative control, untransfected HEK293) or cells containing pLux_{AB} co-transfected with either pLux_{CDEfrp:WT} or pLux_{CDEfrp:CO}. Cells were grown over a 96 hour period until 80% confluent, representing normal passage conditions. Values are the average of three trials and are reported with the standard error of the mean.

FIGS. 4A-4I show the in vitro bioluminescent imaging of lux cassette containing cells. pLux_{CDEfrp:CO}/pLux_{AB} containing (CO), pLux_{CDEfrp:WT}/pLux_{AB} containing (WT), and untransfected negative control (NEG) HEK293 cells were plated in 24-well tissue culture plates and integrated for (FIG. 4A) 10 sec, (FIG. 4B) 1 min, (FIG. 4C) 5 min, (FIG. 4D) 10 min, (FIG. 4E) 15 min, and (FIG. 4F) 30 min. Bioluminescence from cells co-transfected with pLux_{CDEfrp:CO}/pLux_{AB} was distinguishable from background in the presence of untransfected cells after 10 sec and showed no increase in background detection even after a 30 min integration time. Long term in vitro expression (FIG. 4G) demonstrates the temporal longevity of the signal without exogenous amendment. The minimum detectable number of bioluminescent cells was determined (FIG. 4H) by plating a range of cell concentrations in equal volumes of media in triplicate (downward columns) in an opaque 24-well tissue culture plate. The minimum number of cells that could be consistently detected was approximately 20,000. Average radiance was shown to correlate with plated cell numbers (FIG. 4I), yielding an R² value of 0.95275.

FIGS. 5A-5E show in vivo bioluminescent imaging using HEK293 cell expression of mammalian-adapted lux. FIG. 5A, HEK293 cells containing the mammalian adapted pLux_{CDEfrp:WT}/pLux_{AB} cassette (Full) were subcutaneously injected into nude mice and imaged. Detection occurred nearly immediately (<10 sec) post-injection and remained visible up to the 60 min time point of the imaging assay. HEK293 cells containing only the pLux_{AB} plasmid (luxAB) were subcutaneously injected into the same mouse as a negative control. Note that the automatic scaling of signal intensity differs among images, therefore creating the false appearance that image intensity is decreasing after the 10 min post-injection time point when in fact it continually increases as shown in FIG. 5B. FIG. 5C, comparison of mammalian-adapted lux-based bioluminescence from HEK293 cells versus published data on the expression of FLuc and RLuc tagged cells over the 60 min course of the assay. FIG. 5D, upon termination of the assay 60 min post injection, the bioluminescent signal from HEK293 cells expressing the full complement of lux genes was detectable using an integration time as low as 30 sec. FIG. 5E, subcutaneous injection of HEK293 cells containing pLux_{CDEfrp:WT}/pLux_{AB} at concentrations ranging from 500,000 to 25,000 cells in 100 μl volumes of PBS demonstrated a tested lower limit of detection of 25,000 cells using a 10 min integration time. MPI, minutes post injection.

FIGS. 6A-6F show the comparison of in vitro imaging results. Pseudocolor representation of the bioluminescent or fluorescent flux from cell concentrations ranging from 1 million (1M) to several thousand (K) to approximately single cell levels (NEG=negative control wells) stably transfected with (FIG. 6A) holux, (FIG. 6C) Luc, or (FIG. 6E) GFP. Lines indicate the combination of two separate runs, each represented by the corresponding color scale on the right or left side of the figure. The box in (FIG. 6E) indicates wells containing equal numbers of untransfected HEK293 cells to determine levels of background autofluorescence. Note that autoscaling of the pseudocolor image assigns brighter colors and larger areas to the larger population sizes of low level detection experiments although their scale indicates overall lower levels of flux compared to larger population sizes. Average bioluminescent or fluorescent flux dynamics for the (FIG. 6B) holux, (FIG. 6D) Luc, and (FIG. 6F) GFP containing cell populations of ˜1×10⁶ cells over a 24 h period demonstrate the differences in signal intensity over time.

FIG. 7 shows the detection of 10,000 lux-tagged HEK293 cells was not possible at statistically significant levels. Despite presenting an intermittently detectable pseudocolor image, a population of ˜10,000 holux expressing cells could not be statistically differentiated from background light detection. Boxes represent the mean values of three trials, reported with overlapping standard error of the means.

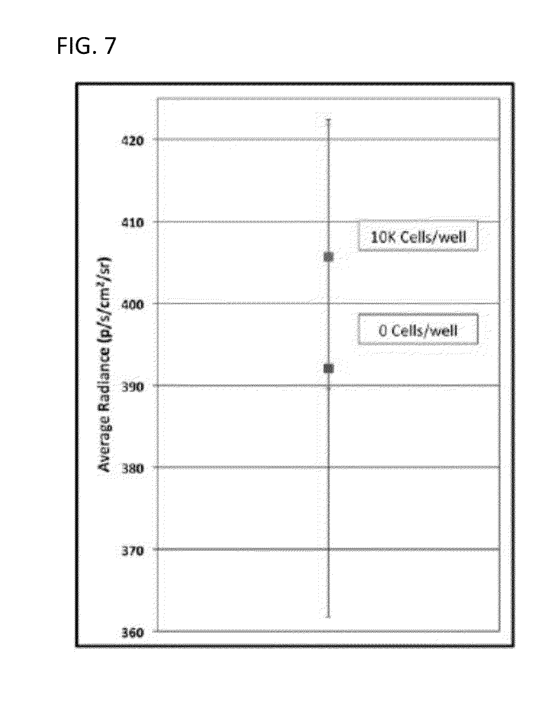

FIGS. 8A and 8B show the minimum population size detection of Luc-tagged HEK293 cells. Short integration times (˜1 sec) are required to prevent saturation of the CCD camera when using a Luc-based reporter system due to its high levels of bioluminescent flux following D-luciferin amendment. However, at integration times of 1 sec (FIG. 8A) it is not possible to differentiate Luc expressing cell populations below ˜250 cells from background light detection. Increasing the integration time to ˜10 sec (FIG. 8B) in the absence of larger population sizes to prevent camera saturation allows for detection down to ˜50 cells. Boxes represent the mean values of three trials, reported with the standard error of the mean.

FIG. 9 shows the minimum population size detection of GFP-tagged HEK293 cells. Cells expressing GFP were visible down to population sizes of ˜5×10⁵ cells. Boxes represent the mean values of three trials, reported with the standard error of the mean.

FIGS. 10A-10F show the comparison of in vivo bioluminescence for holux and Luc cells. The bioluminescent signal following subcutaneous injection of holux-expressing cells (FIG. 10A) remains relatively stable following injection and is detectable (FIG. 10B) down to a minimum of ˜25,000 cells. Signal dynamics are significantly altered (FIG. 10C), but of approximately the same strength following intraperitoneal injection. Total flux from subcutaneous injection of Luc-expressing cells (FIG. 10D) is significantly higher, and as such is detectable (FIG. 10E) down to ˜2,500 cells. Bioluminescent output from intraperitoneal injected Luc-expressing cells (FIG. 10F) expressed peak flux immediately following D-luciferin injection, but then quickly diminished over the remainder of the assay.

FIGS. 11A-11D shows a comparison of pseudocolor images of subcutaneously and intraperitonealy injected holux and Luc Cells. Subcutaneously injected (FIG. 11A) holux or (FIG. 11B) Luc expressing cells are capable of presenting relatively similar images despite the large differences in total flux from each reporter system if the integration time is increased from 1 sec (Luc) to 60 sec (holux). Similar increases must be made to maintain uniform representative detection following intraperitoneal injection of the (FIG. 11C) holux and (FIG. 11D) Luc cells as well, with the holux system requiring a 60 sec integration time to achieve similar pseudocolor patterning as a 10 sec integration of the Luc system.

FIG. 12 shows bioluminescent production following treatment with varying levels of doxycycline. Cells treated with 100 ng doxycycline/ml produced significantly greater bioluminescent flux (p<0.05) than untreated cells at all time points except for 5 h post treatment, while cells receiving 10 ng doxycycline/ml produced greater bioluminescent flux only intermittently.

FIG. 13 shows a bioluminescent profile of constitutively bioluminescent HEK293 cells following decanal treatment. Treatment with 0.1% decanal leads to immediate reductions in bioluminescent output, while treatment with lower concentrations produces more subtle reductions in output. Only treatment with 0.1% and 0.01% decanal adversely effected bioluminescent production, while no treatment levels surveyed were capable of increasing bioluminescent production.

FIG. 14 shows supplementation assays demonstrating the functionality of the luxCDEfrp genes in the mammalian cell environment

FIG. 15A-15E shows a pseudocolor representation of bioluminescent flux (photons/sec/cm²/steridian) emanating from zebrafish microinjected with either the luxAB genes (FIGS. 15A and 15D) or the luc gene (FIGS. 15B and 15E) at 3-5 and 7 days post fertilization (dpf). FIGS. 15C and 15F, noninjected control fish.

FIG. 16 is Table 2. Detection of lux-expressing HEK293 cells over time. Larger population sizes of lux-expressing cells were visible sooner following plating. Green boxes represent time points where the indicated cell population was significantly distinguishable from background. Red hatched boxes represent time points where the indicated cell population was not significantly distinguishable from background light detection.

FIG. 17 is Table 3. Detection of Luc-expressing HEK293 cells over time. Larger populations of Luc-expressing cells were visible over longer periods of time following the addition of D-luciferin. Due to the highly dynamic nature of Luc expression, readings are reported at 10 min intervals. Green boxes represent time points where the indicated cell population was significantly distinguishable from background. Red hatched boxes represent time points where the indicated cell population was not significantly distinguishable from background light detection significantly distinguishable from background.

FIG. 18 is Table 4. Detection of GFP-expressing HEK293 cells over time. GFP-expressing cells could be significantly differentiated from background fluorescence detection at all time points following plating when greater than ˜5×10⁵ cells were present. Detection of ˜5×10⁵ cells became possible 9 hr after plating, while detection of less than ˜5×10⁵ cells was not possible at any of the time points surveyed. Green boxes represent time points where the indicated cell population was able to be significantly distinguishable from background. Red hatched boxes represent time points where the indicated cell population was not significantly distinguishable from background light detection.

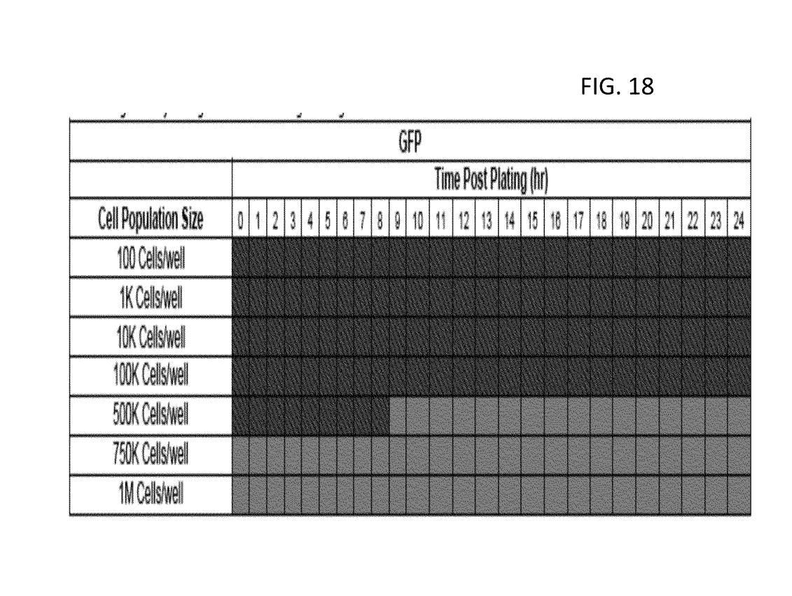

FIG. 19 is Table 5. Summary of comparisons between the holux, Luc, and GFP reporter systems under in vitro and in vivo imaging conditions.

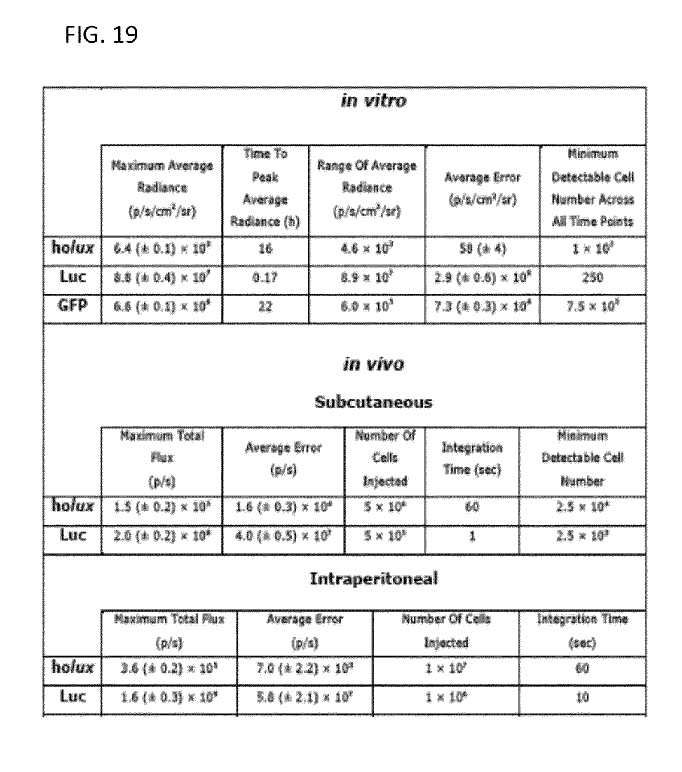

FIG. 20 is Table 6. Regulation of luxC gene transcription in response to doxycycline treatment. Cell lines demonstrating a significant (p<0.05) reduction in CT value between both negative control and 10 ng/ml doxycycline treatment and negative control and 100 ng/ml doxycycline treatment are indicated with the * symbol. The cell line selected for further testing is designated by the ** symbol.

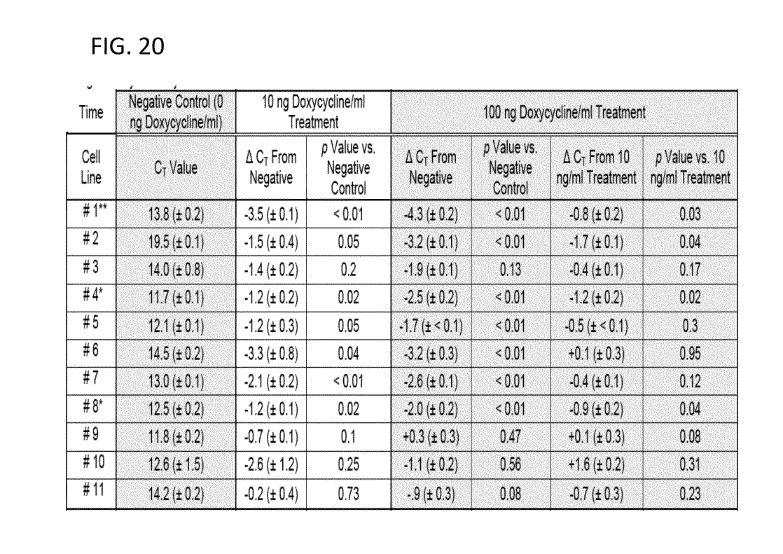

FIG. 21 is Table 7. Detection of significantly significant changes in bioluminescent production following doxycycline treatment. HEK293 cells containing promoter sequences capable of regulating luxC and luxE gene expression in response to doxycycline levels can be used to report on exposure to increased levels of doxycycline in the media. Green boxes indicate time points, where significant (p<0.05) up regulation of bioluminescent production was detected. Hatched red boxes indicate that no significant (p>0.05) increase in bioluminescent production was detected.

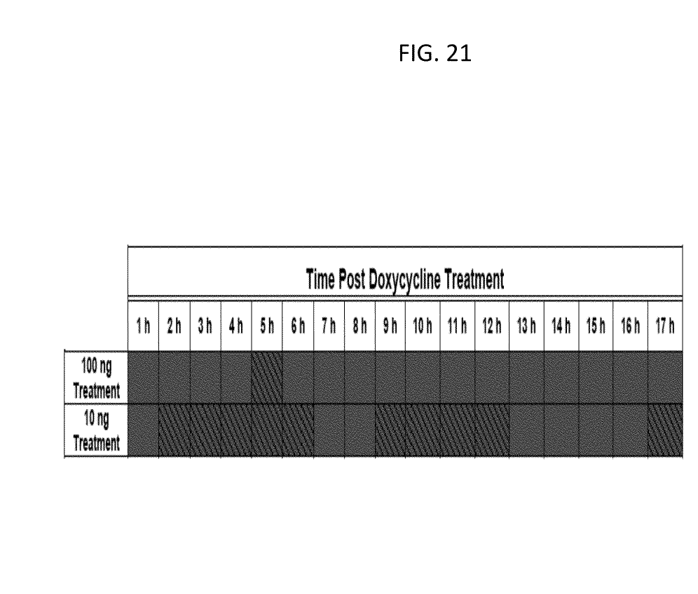

FIG. 22 is Table 8. Detection of significantly different changes in bioluminescent production following doxycycline treatment. Hashed red boxes indicate bioluminescent profiles similar to control (p>0.05). Green boxes represent bioluminescent profiles lower than untreated control cells (p<0.05). At concentrations below 0.01% decanal it was not possible to differentiate the luminescent profile from that of the untreated control cell line. Intermittent deviation in bioluminescent flux was detected beginning at 40 min post decanal addition and became constant after 4 h at a concentration of 0.01%, while treatment with 0.1% decanal was able to be differentiated from control at all time points surveyed.

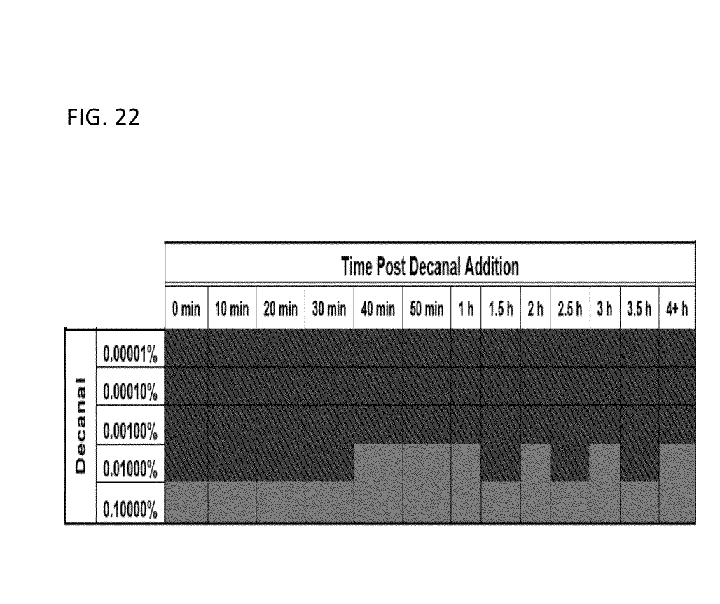

FIG. 23 is Table 9. Bioluminescent expression from in vitro expression of luxA and luxB genes linked by 2A elements is consistently higher than that of IRES linked luxA and luxB genes.

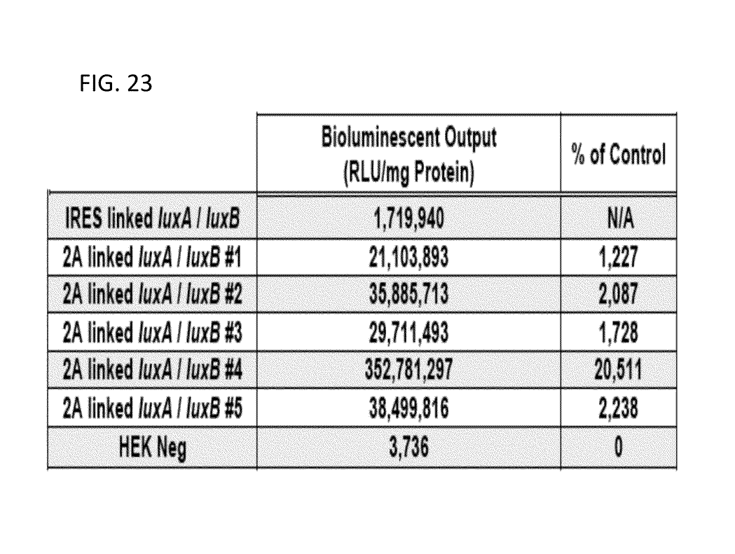

### DETAILED DESCRIPTION OF SEVERAL EMBODIMENTS

### I. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). The Zebrafish: Biology. Methods in Cell Biology. Volume 59, and Detrich et al. (1998) The Zebrafish: Genetics and Genomics. Methods in Cell Biology. Volume 60 for techniques of zebrafish maintenance, mutagenesis, transgenesis and mapping.

The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of terms are provided:

Animal: A living multi-cellular vertebrate organism, a category that includes, for example, mammals and fish, such as zebrafish.

Codon Optimization: A strategy in which codons within a cloned gene—ones not generally used by the host cell translation system—are changed by mutagenesis to the preferred codons of the host organism, without changing the amino acids of the synthesized protein.

Degenerate variant and conservative variant: A polynucleotide encoding a polypeptide that includes a sequence that is degenerate as a result of the genetic code.

For example, a polynucleotide encoding a disclosed bacterial luciferase gene, such as a luxA, luxB, luxC, luxD, luxE or rtf gene includes a sequence that is degenerate as a result of the genetic code. There are 20 natural amino acids, most of which are specified by more than one codon. Therefore, all degenerate nucleotide sequences are included as long as the amino acid sequence of the peptide or protein encoded by the nucleotide sequence is unchanged.

One of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitutions providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Not all residue positions within a protein will tolerate an otherwise “conservative” substitution. For instance, if an amino acid residue is essential for a function of the protein, even an otherwise conservative substitution may disrupt that activity.

Expression: Translation of a nucleic acid into a protein. Proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter, the cytomegalovirus promoter) can be used. Promoters produced by recombinant DNA or synthetic techniques may also be used to provide for transcription of the nucleic acid sequences.

Isolated: An “isolated” biological component (such as a vertebrate cell) has been substantially separated, produced apart from, or purified away from other biological components, such as cells of the organism in which the component naturally occurs. Nucleic acids, peptides and proteins that have been “isolated” thus include nucleic acids and proteins purified by standard purification methods. The term also embraces nucleic acids, peptides, and proteins prepared by recombinant expression in a host cell as well as chemically synthesized nucleic acids.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Such polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. These techniques can be used to alter the coding sequence without altering the amino acid sequence that is encoded. Unless otherwise specified, any reference to a nucleic acid molecule includes the reverse complement of the nucleic acid. Any nucleic acid written to depict only a single strand encompasses both strands of a corresponding double-stranded nucleic acid. Additionally, reference to the nucleic acid molecule that encodes a specific protein, or a fragment thereof, encompasses both the sense strand and its reverse complement. The present invention also provides a vector comprising any of the nucleic acids set forth herein. These include vectors for expression in both eukaryotic and prokaryotic host cells, either in vitro, in vivo or ex vivo.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or, expression of the coding sequence. Generally, operably linked DNA sequences are contiguous and, where necessary to join two protein-coding regions, in the same reading frame.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g.; by genetic engineering techniques.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the interne, for use in connection with the sequence analysis programs blastp, blastn, blastx, tblastn and tblastx. A description of how to determine sequence identity using this program is available on the NCBI website on the interne.

Transgenic: A transgenic cell or animal contains one or more transgenes within its genome. A transgene is a DNA sequence integrated at a locus of a genome, wherein the transgenic DNA sequence is not otherwise normally found at that locus in that genome. Transgenes may be made up of heterologous or homologous DNA sequences.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and, Harlow and Lane, Using Antibodies: A Laboratory Manual, Cold Spring Harbor Laboratory Press, 1999. In addition, the materials, methods, and examples are illustrative only and not intended to be limiting.

Zebrafish: The zebrafish, Danio rerio, is a tropical freshwater fish belonging to the minnow family.

### II. Description of Several Embodiments

A. Introduction

In vivo optical imaging is becoming increasingly utilized as a method for modern biomedical research. This process, which involves the non-invasive interrogation of animal or cellular systems subjects, for example using light emitted either naturally from a luciferase protein or following excitation of a fluorescent protein or dye, has been applied to the study of a wide range of biological processes such as gene function, drug discovery and development, cellular trafficking, protein-protein interactions, and especially tumorigenesis and cancer treatment. While the detection limits and resolution of charge coupled devices (CCDs) has increased greatly in recent years, there have been relatively few introductions of improved imaging compounds that function as light production centers within an animal subject in vivo.

Generally, the currently available imaging compounds can be divided into two classes: those containing luciferase proteins (capable of producing bioluminescent light without exogenous excitation) and those containing fluorescent compounds (dyes or proteins that require an initial excitation followed by emission at a given wavelength). For mammalian-based whole animal imaging, fluorescent compounds are limited due to high levels of background fluorescence from endogenous biological structures upon excitation. Although dyes have been developed and employed that fluoresce in the near infrared wavelengths where light absorption is lowest in mammalian tissues, they can become increasingly diffuse during the process of cellular division, negating their usefulness in long term monitoring studies. In contrast, luciferase proteins are highly amenable towards in vivo optical imaging because they produce a controllable light signal in cells with little to no background bioluminescence, thus allowing for remarkably sensitive detection.

Historically the luciferase proteins used have been based on beetle luciferases (e.g., firefly or click beetle luciferase) or marine aequorin-like proteins (those that utilize coelenterazine), these each possess disadvantages when applied to whole animal studies. For example, the popular firefly luciferase protein is heat labile when incubated under whole animal imaging conditions, and can display a half life as short as 3 min in its native state at 37° C. Coelenterazine-stimulated luciferases are similarly handicapped in regards to long-term monitoring, as it has been reported that their rapid uptake of coelenterazine necessitates prompt imaging following substrate injection. Applications of both these luciferase systems also suffer from the drawback that they require addition of an exogenous substrate to produce a detectable light signal.

Bioluminescent bacteria are the most abundant and widely distributed of the light emitting organisms on Earth and can be found in both aquatic (freshwater and marine) and terrestrial environments. Despite the diverse nature of bacterial bioluminescence, the majority of these organisms are classified into three genera: Vibrio, Photobacterium, and Photorhabdus (Xenorhabdus).

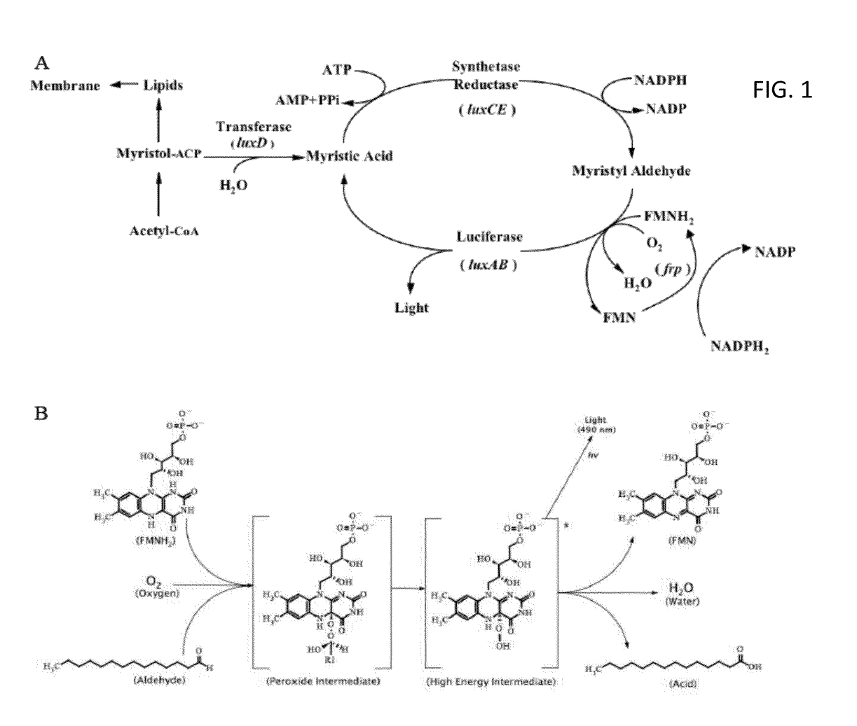

The bacterial bioluminescence reaction is the result of two proteins, LuxA and LuxB, that work together to produce light from the oxidation of a long chain fatty aldehyde in the presence of reduced riboflavin phosphate (FMNH₂) and oxygen, while the remaining proteins in the lux operon, LuxC, LuxD, and LuxE, function to regenerate the aldehyde substrate required for this reaction (FIG. 1A).

While the bacterial luciferase protein is all that is required to generate light in the presence of its required substrates, it is often beneficial for investigators to express other genes from the operon in order to supply the luciferase with the substrates required for its autonomous function. To accomplish this, it is necessary to co-express the luxC, luxD, and luxE genes. The products of these genes assemble into a multi-enzyme complex and are responsible for biosynthesis of myristyl aldehyde using components already present in the cell, thus negating the requirement to supply an aldehyde substrate exogenously.

The luxD gene encodes a transferase protein and is the first to act in the aldehyde biosynthesis pathway. It is responsible for the transfer of an activated fatty acyl group to water, forming a fatty acid. During the course of this reaction the enzyme itself becomes acylated. The newly formed fatty acid is next passed off to the luxC gene product, which activates the acid by attaching AMP from a molecule of ATP, thereby creating a fatty acyl-AMP that remains tightly bound to the enzyme. The fatty acyl-AMP is then transferred to the luxE gene product via transfer of the acyl group. This protein acts as a reductase and catalyzes the reduction of the fatty acyl-AMP to aldehyde using NADPH to supply the required reducing power. This allows for the in vivo generation of the aldehyde substrate. Because the organism naturally supplies the remaining FMNH₂ and oxygen substrates, the co-expression of these genes represents the minimum requirement for allowing the lux system to operate in a fully autonomous fashion. Oxygen and FMNH₂ are naturally occurring products within the cell while the bacterial luxCDE gene products produce and regenerate the aldehyde substrate using endogenous aliphatic compounds initially targeted to lipid biogenesis. To produce light, the luciferase protein first binds FMNH₂, followed by O₂, and then the synthesized aldehyde. This allows the lux cassette to utilize only endogenous materials to form an intermediate complex that then slowly oxidizes to generate light at a wavelength of 490 nm as a byproduct (Meighen 1991). The overall reaction can be summarized as: FMNH₂+RCHO+O₂>FMN+H₂O+RCOOH+hv490 nm.

Realizing the distinct advantages bacterial luciferase would afford as a eukaryotic reporter, many groups have attempted to express the luciferase (luxAB) component of the lux system using either fusion proteins or multiple plasmids, but with minimal success over the last twenty years. Although the use of lux in the study of bacterial infection of a mammalian host has been demonstrated using whole animal imaging its functionality has not been demonstrated outside of a bacterial host until now.

Since its characterization, the definitive shortcoming of the bacterial luciferase (lux) bioluminescent reporter system has been its inability to express at a functional level in the eukaryotic cellular background. While recent developments have allowed for lux function in the lower eukaryote Saccharomyces cerevisiae, they have not provided for autonomous function in higher eukaryotes capable of serving as human biomedical proxies.

### Polynucleotides Encoding Lux Operons

As disclosed herein a modified bacterial luciferase gene cassette can be expressed in mammalian cells in culture or in whole animal BLI without the use of exogenous substrates or coincident infection with a bacterial host, thus overcoming the limitations imposed by currently available luciferase-based BLI assays. Setting the bacterial bioluminescence system apart from other bioluminescent systems such as firefly luciferase and aequorin is its ability to self-synthesize all of the substrates required for the production of light. While the luciferase component is a heterodimer formed from the products of the luxA and luxB genes, its only required substrates are molecular oxygen, reduced riboflavin phosphate (FMNH₂), and a long chain aliphatic aldehyde.

To fully exploit the advantages of bacterial luciferase, all five genes (luxCDABE) of the lux operon must be expressed simultaneously. As disclosed herein, it is demonstrated that codon-optimized, poly-bicistronic expression of the full lux cassette produces all of the products required for autonomous bioluminescent production in a mammalian background. It is further demonstrated that cells expressing the full lux cassette can be applied towards whole animal BLI without the need for substrate addition, thus overcoming the limitations imposed by currently available luciferase-based whole animal BLI probes.

Disclosed herein are codon optimized polynucleotides encoding Lux operons. The nucleic acids may be in the form of RNA or in the form of DNA (e.g., cDNA, genomic DNA, and synthetic DNA). The nucleic acids are codon optimized for expression in a vertebrate cell, such as a mammalian cell or a fish cell, such as a zebrafish cell. The DNA may be double-stranded or single-stranded. In some examples described herein, nucleic acids encoding LuxA, LuxB, LuxC, LuxD, and LuxE were derived from wild-type P. luminescens. Nucleic acid sequences which encode native P. luminescens LuxA, LuxB, LuxC, LuxD, and LuxE proteins are listed in Genbank as accession numbers AF403784, M62917, M55977, M90092, and M90093, respectively and are hereby incorporated herein in their entirety as available May 23, 2011. The amino acid sequences of native P. luminescens LuxA, LuxB, LuxC, LuxD, and LuxE proteins are listed in Genbank as accession numbers AAK98554, AAK98555, AAK98552, AAK98553, and AAK98556, respectively and are hereby incorporated herein in their entirety as available May 23, 2011. Nucleic acids encoding LuxA, LuxB, LuxC, LuxD, and LuxE derived from other strains or organisms can also be used so long as they can be expressed in vertebrate cells to generate luminescence. For example, nucleic acids encoding LuxA, LuxB, LuxC, LuxD, and LuxE proteins from Vibrio harveyi, P. luminescens, Photobacterium phosphoreum, Photobacterium leiognathi, and Shewanella hanedai can also be used in the compositions and methods disclosed herein. Also encompassed by this disclosure are nucleic acid molecules encoding active fragments, analogs and derivatives of LuxA, LuxB, LuxC, LuxD, and LuxE proteins and those that encode mutant forms of these proteins or non-naturally occurring variant forms of these proteins.

In some examples, the disclosed nucleic acids include frp nucleic acids encoding NAD(P)H-flavin oxidoreductase protein (FMN oxidoreductase, also known as NAD(P)H-FMN oxidoreductase). NAD(P)H-flavin oxidoreductases (flavin reductases (FR)) are a class of enzymes that catalyze the reduction of flavin by NAD(P)H. The complete luciferase enzyme is a flavin monooxygenase that binds a reduced flavin molecule (FMNH₂) as a specific substrate. Bioluminescence levels in eukaryotic cells may therefore be increased by increasing expression of FMN oxidoreductase in the cells. The FMN oxidoreductase from V. harveyi (See Genbank accession number AAA21331 and U08996, which are specifically incorporated herein as available May 23, 2011), V. fischeri, Escherichia coli, and Helicobacter pylori can be used.

In some examples, the nucleic acids encoding LuxA, LuxB, LuxC, LuxD, LuxE and/or frp proteins are codon-optimized for expression in a vertebrate cell, such as a mammalian or fish cell. Thus in some examples, or more, of LuxA, LuxB, LuxC, LuxD, LuxE and/or frp proteins is codon optimized. Codon optimization has become routine in the art based on available codon usage tables.

A nucleic acid encoding LuxA, LuxB, LuxC, LuxD, LuxE and/or frp proteins can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Methods for the manipulation and insertion of the nucleic acids of this disclosure into vectors, such as viral vectors, are well known in the art (see for example, Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

The polynucleotides encoding a LuxA, LuxB, LuxC, LuxD, LuxE and/or frp protein includes recombinant DNA and or RNA which is incorporated into one or more vectors, autonomously replicating plasmids or virus, or into the genomic DNA of a prokaryote or eukaryote, or which exists as a separate molecule (such as a cDNA) independent of other sequences. In some examples, the polynucleotide sequences encoding a LuxA, LuxB, LuxC, LuxD, LuxE and/or frp protein are present on more than one vector, autonomously replicating plasmids or virus, such as 2, 3, 4, 5 or even six vectors, autonomously replicating plasmids or viruses. Polynucleotide sequences encoding a LuxA, LuxB, LuxC, LuxD, LuxE and/or frp protein can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Any suitable vector that includes a replication system and sequences that are capable of transcription and translation of a polypeptide-encoding sequence in a given host cell may be used. Expression vectors may include-regulatory elements that facilitate expression of a polypeptide in a host cell. Examples of regulatory elements include promoters, enhancers, initiation sites, polyadenylation (polyA) tails, internal ribosome entry site (IRES) elements, proteins, any of a number of promoters suitable for use in the selected host cell may be employed. For example, constitutive promoters of different strengths can be used to express the LuxA, LuxB, LuxC, LuxD, LuxE and/or frp. Inducible promoters may also be used in compositions and methods of the invention. To achieve regulated expression of LuxA, LuxB, LuxC, LuxD, LuxE and/or frp proteins in mammalian cells, a constitutive cytomegalovirus (CMV) promoter or human elongation factor 1″ (EF-1″) promoter is preferred, however, any promoter known to function in mammalian cells may be used. To increase levels of bacterial luciferase proteins, nucleic acids encoding these proteins are operably linked to any of a number of enhancers suitable for use in mammalian (e.g., human) cells. One example of an enhancer that may be useful is the SP163 site, an untranslated, region in the mouse genome that has been shown to increase translation several-fold when placed upstream of genes in mammalian cells.

To facilitate expression of a nucleic acid, the nucleic acid may be operatively linked to an IRES element. IRES elements allow ribosomes to bind directly at an AUG start codon rather than requiring initial recognition at the 5′ cap site and subsequent scanning for the start site. If the AUG start site is located within the open reading frame, translation can be initiated internally and a monocistronic mRNA essentially becomes multiply-cistronic. The insertion of an IRES fragment between lux (e.g., luxA, luxB, luxC, luxD, luxE) nucleic acids facilitates bicistronic synthesis of Lux proteins. Similarly, insertion of an IRES fragment between lux (e.g., luxA, luxB, luxC, luxD, luxE) and frp nucleic acids facilitates bicistronic synthesis of Lux and FMN oxidoreductase proteins. In some examples, 2A elements are used in place of an IRES fragment.

In some embodiments, the nucleic acids encoding one or more of LuxA, LuxB, LuxC, LuxD, LuxE and/or frp are operable linked to a regulatory element that is a pollutant-inducible DNA response element. In some examples, the pollutant-inducible DNA response element is a modular enhancer unit or response element selected from the group consisting of the metal response element (MRE), the aromatic hydrocarbon response element (AHRE), the estrogen response element (ERE), the androgen response element (ARE), the electrophile response element (EPRE), and the retinoic acid response elements (RARE, RXRE). The response element controls is thus able to control the expression of one or more of LuxA, LuxB, LuxC, LuxD, LuxE and frp elements by controlling the transcription.

Some of the enhancer regions (DNA motifs) that been characterized include the metal response element (MRE), the aromatic hydrocarbon response element (AHRE), the estrogen response element (ERE), the androgen response element (ARE), the electrophile response element (EPRE), and two retinoic acid response elements (RARE, RXRE). Heavy metals such as cadmium, zinc or mercury turn on particular genes via the MRE. Dioxin, polychlorinated biphenyls (PCBs), and benzpyrene generated in combustion processes turn on some genes via the AHRE. Environmental and natural estrogens turn on specific genes via the ERE. Environmental and natural androgens turn on specific genes via the ARE. Oxidants such as bleaching agents and hydrogen peroxide turn on distinct genes via the EPRE. Certain retinoids turn on certain genes via the RARE and RXRE.

Typically, inducible response elements consist of a core consensus sequence, which usually is influenced by its flanking sequences and/or nearby multiple response elements in causing maximal induction. Aromatic hydrocarbon response elements (AHREs) respond to a wide variety of polycyclic hydrocarbons and halogenated planar molecules such as 2,3,7,8-tetrachlorodibenzo-p-dioxin (TCDD; dioxin) and polychlorinated biphenyls (PCBs) as well as polychlorinated dibenzo-p-dioxin (PCDD), polychlorinated dibenzofuran (PCDF), and polychlorinated di-aromatic hydrocarbon (PCDH), a kind of polyaromatic hydrocarbon (PAH). Quinones and a wide variety of other potent electrophilic oxidants activate electrophile response elements (EPREs). Metal response elements (MREs) respond to heavy metals such as mercury, copper, nickel, cadmium and zinc. Estrogen response elements (EREs) are upregulated by estrogens and other environmentally important endocrine disruptors. Androgen response elements (AREs) are upregulated by androgens and other environmentally important endocrine disruptors. Retinoic acid response elements (RAREs) and retinoid X receptor response elements (RXREs) respond to 9-cis-retinoic acid and other retinoids.

Aromatic hydrocarbon response element (AHRE). Ligands for the Ah receptor (AHR) activate the AHRE and many adverse biological effects including immunosuppression, teratogenesis, tumor promotion, endocrine disruption, and cardiovascular disease. Upon binding ligand, the AHR translocates to the nucleus and binds to AHRE motifs located in the promoter region of the mammalian CYP1A1 and probably more than a dozen other genes. Halogenated and nonhalogenated polycyclic hydrocarbons (e.g. polychlorinated biphenyls, TCDD, benzo[a]pyrene) are ligands for the AHR and, thus, activate genes via AHREs. An example of this system is U.S. Pat. Nos. 5,854,010 and 5,378,822, incorporated by reference.

Electrophile response element (EPRE). Also called “antioxidant response element” (ARE), the EPRE is activated following treatment with potent oxidants and electrophiles, leading to the induction of numerous stress-inducible genes. Electrophilic compounds and metabolites that activate EPREs also react with nucleophilic centers on macromolecules and are involved in mutagenesis, carcinogenesis and aging. Inducing agents include not only reactive hydrogen peroxide, phenols and quinones but also metabolites of phase I metabolism such as oxygenated benzo[a]pyrene or naphthoflavone. EPRE sequences have been found upstream of phase II drug-metabolizing genes and other genes that respond to oxidative stress.

Metal response element (MRE). MREs were first identified upstream of the mouse metallothionein (Mt1, Mt2) genes. Heavy metal cations that induce via the MRE include cadmium, zinc, mercury, cobalt and nickel. Several heavy metals are potent electrophiles, thus activating the EPRE as well as the MRE, leading to mutagenesis and carcinogenesis. Induction of genes via MREs occurs upon exposure to heavy metals such as cadmium, silver, copper, cobalt, mercury, and nickel; zinc and heavy metal toxicity has been demonstrated in virtually every organ system.

Estrogen response element (ERE). The estrogen receptor (“ER”) binds a number of estrogenic compounds and forms a transcription complex with the ERE as a homodimer. Environmental and dietary “endocrine disruptors” bind (to varying degrees) to the ER and are purported to disrupt normal cellular signaling and lead to reproductive tissue abnormalities and/or cancer. Several environmental and pharmaceutical chemicals exhibit varying degrees of estrogenicity including diethylstilbestrol, tamoxifen, dietary phytoestrogens, phthalate plasticizers, insecticides (e.g. p,p′-DDT, p,p′-DDE, dieldrin, methoxychlor, toxaphene, endosulfan), and 4-nonylphenol, bis-phenol-A and kepone.

Androgen response element (ARE). Many anthropogenic and naturally occurring chemicals are suspected to cause endocrine disruption in humans and wildlife, including androgens and androgen-mimicking chemicals. The agonist activity of androgens, required for normal sexual development in males, is mediated by the androgen receptor protein (AR). When the AR binds to an androgenic compound, the complex moves to the nucleus, whereupon it binds to AREs that mediate specific gene expression. Numerous biological functions are mediated by this system, including facial hair development, increased muscle mass, spermatogenesis, and vocal cord enlargement, but when present in excess, androgens pose potential problems for vertebrates. Evidence suggests that in wildlife, environmental exposure to low levels of androgen-mimicking chemicals may alter sex ratios and lead to endocrine dysfunction. Several environmental and pharmaceutical chemicals exhibit varying degrees of androgenic activity including testosterone, dihydrotestosterone (DHT), androstenedione, fluoxymesteron, and norgestrel.

Retinoic acid and retinoid X response elements (RAREs, RXREs). Both retinoic acid receptors (RARs) and retinoid X receptors (RXRs) bind with high affinity to 9-cis-retinoic acid but show striking differences in their affinity for other retinoids. Many retinoic acid analogues have been developed as therapeutic and chemopreventive agents and bind preferentially to specific RAR and/or RXR isoforms activating RAREs and RXREs. The popular insecticide methoprene has been found to be a potent RXR agonist. An imbalance in the normal levels of retinoic acid (vitamin A) and/or its derivatives can cause striking deformities in limbs and other organs during embryonic development or regeneration. Environmental retinoids have been implicated in frog deformities in the Great Lakes Area where a powerful teratogen appears to exist in groundwater and well water.

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation. When the host is a eukaryote, such methods of transfection of DNA as calcium phosphate coprecipitates, conventional mechanical procedures such as microinjection, electroporation, insertion of a plasmid encased in liposomes, or viral vectors can be used.

Disclosed are vertebrate cells (such as mammalian or fish cells) transformed with the disclosed polynucleotides encoding a LuxA, LuxB, LuxC, LuxD, LuxE and/or frp protein. DNA sequences encoding a LuxA, LuxB, LuxC, LuxD, LuxE and/or frp protein can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art. The disclosed cells can be used as autonomous biosensors to detect for example toxic chemicals. For example by contacting the cells with an environmental sample.

In some embodiments, a disclosed cell, is exposed to a sample to be tested under conditions permitting expression of the reporter gene and detected for reporter gene expression. Generally, the contaminant to be detected is selected from the group consisting of polyaromatic hydrocarbons, electrophilic oxidants heavy metals, endocrines, and retinoids. Preferably, the contaminant to be detected is selected from the group consisting d-2,3,7,8-tetrachlorodibenzo-p-dioxin, dioxin, polychlorinated biphenyls, quinones, mercury, copper, nickel, cadmium, zinc, estrogens, retinoic acid and 9-cis-retinoic acid.

A number of viral vectors have been constructed, that can be used to express the disclosed mutant EPO polypeptides, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Viral., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell. Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell. Biol., 5:431-437; Sorge et al., 1984, Mol. Cell. Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Nucleic acid molecules encoding LuxA, LuxB, LuxC, LuxD, LuxE or frp can be prepared by chemical peptide synthesis. Nucleic acid molecules encoding LuxA, LuxB, LuxC, LuxD, LuxE or frp can be prepared by cloning techniques. Examples of appropriate cloning and sequencing techniques, and instructions sufficient to direct persons of skill through many cloning exercises are found in Sambrook et al., Molecular Cloning: A Laboratory Manual (2nd Ed.), Vols. 1-3, Cold Spring Harbor Laboratory (1989), Berger and Kimmel (eds.), Guide to Molecular Cloning Techniques, Academic Press, Inc., San Diego Calif. (1987), or Ausubel et al. (eds.), Current Protocols in Molecular Biology, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH® laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), INVITROGEN™ (San Diego, Calif.), and Applied Biosystems (Foster City, Calif.), as well as many other commercial sources known to one of skill.

The transfer of DNA into eukaryotic, in particular human, or other mammalian cells, is now a conventional technique. The vectors are introduced into the recipient cells as pure DNA (transfection) by, for example, precipitation with calcium phosphate (Graham and vander Eb, 1973, Virology 52:466) or strontium phosphate (Brash et al., Mol. Cell. Biol. 7:2013, 1987), electroporation (Neumann et al., EMBO J. 1:841, 1982), lipofection (Feigner et al., Proc. Natl. Acad. Sci. USA 84:7413, 1987); DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-7, 1980), or pellet guns (Klein et al, Nature 327:70., 1987). Alternatively, the cDNA can be introduced by infection with virus vectors. Systems are developed that use, for example, retroviruses (Bernstein et al., Gen. Engrg. 7:235, 1985), adenoviruses (Ahmad et al., J. Virol. 57:267, 1986), or Herpes virus (Spaete et al., Cell 30:295, 1982).

### Zebrafish Reporter

Transgenic zebrafish are disclosed herein, including zebrafish cells and zebrafish embryos, as well as adult or juvenile zebrafish. The transgenic zebrafish disclosed herein, include one or more polynucleotides encoding a LuxA, LuxB, LuxC, LuxD, LuxE and/or frp protein The transgenic zebrafish can be a transient or a stable transgenic zebrafish. As used herein, transgenic zebrafish refers to zebrafish, or progeny of zebrafish into which an exogenous construct, such as the bacterial lux reporter system has been introduced. A zebrafish into which a construct has been introduced includes fish which have developed from embryonic cells into which the construct has been introduced. An exogenous construct is a nucleic acid that is artificially introduced or was originally artificially introduced into an animal. Fish produced by transfer, through normal breeding, of an exogenous construct (that is, a construct that was originally artificially introduced) from a fish containing the construct are considered to contain an exogenous construct. Such fish are progeny of fish into which the exogenous construct has been introduced.

The progeny of a fish are any fish which are descended from the fish by sexual reproduction or cloning, and from which genetic material has been inherited. In this context, cloning refers to production of a genetically identical fish from DNA, a cell, or cells of the fish. The fish from which another fish is descended is referred to as a progenitor fish. Development of a fish from a cell or cells (embryonic cells, for example), or development of a cell or cells into a fish, refers to the developmental process by which fertilized egg cells or embryonic cells (and their progeny) grow, divide, and differentiate to form an adult fish.

The disclosed transgenic fish are produced by introducing a transgenic construct into cells of a zebrafish, preferably embryonic cells, and most preferably in a single cell embryo. The transgenic construct is preferably integrated into the genome of the zebrafish. However, the construct can also be constructed as an artificial chromosome. The transgenic construct can be introduced into embryonic cells using any technique known in the art or later developed for the introduction of transgenic constructs into embryonic cells. For example, microinjection, electroporation, liposomal delivery and particle gun bombardment can all be utilized to effect transgenic construct delivery to embryonic cells as well as other methods standard in the artt for delivery of nucleic acids to zebrafish embryos or embryonic cells.

Zebrafish containing a transgene can be identified by numerous methods such as probing the genome of the zebrafish for the presence of the transgene construct by Northern or Southern blotting. Polymerase chain reaction techniques can also be employed to detect the presence of the transgene. For example, RNA can be detected using any of numerous nucleic acid detection techniques, such as reverse transcriptase PCR. Alternatively, an antibody can be used to detect the expression. One of skill in the art can also utilize other immunohistochemical techniques available in the art.

Zebrafish oocytes and fertilized eggs are generally transparent and easy to use for microinjection. They hatch in 2-3 days and have a relatively short generation time of 3-4 months. Well-characterized transcription control elements from viruses and mammals are able to direct protein expression in fish cells. More recently, promoter elements isolated from fish species have been analyzed for their capacity to direct protein synthesis in fish cells and transgenic animals.

The zebrafish is an efficient vertebrate model system because of its relatively short reproductive cycle, the large number of progeny that can be produced, and the relatively small space needed to maintain large numbers of offspring at low cost. Zebrafish embryos are also transparent and accessible throughout development, which allows for easy microinjection and other manipulations. Moreover, the zebrafish is becoming a powerful system for genetic analysis with the development of a high-density genome map by the Zebrafish Genome Project.

Relatively simple and reliable methods for the production of transgenic zebrafish have also been developed. Gene transfer into embryos has improved with the use of retroviral vectors and transposons, and the use of border elements has stabilized the expression of transgenes in subsequent generations.

Because it is preferable to assay luminescence or fluorescence in the living intact fish, it is preferable to use zebrafish lines lacking pigmentation. Initial studies with a mutant albino line revealed this line would be difficult due to chronic poor breeding. Alternatively, the golden, long-fin zebrafish (gol/lof) zebrafish line works well because the very long fins are an excellent source of tissue for genotyping and because it has reduced amounts of body pigmentation.

Generally, for the insertion of plasmids into the zebrafish embryo, electroporation or microinjection may be used although the latter tends to be more efficient. Alternatively, transgenic animals can be made using constructs containing the locus control region (LCR) of the mouse Mt1 gene, in order to create an artificial locus. Since, it is often difficult to maintain transgenes through many subsequent generations, insulating border elements, such as the Mt1-LCR, are typically used to stabilize the expression of transgenes in zebrafish for several generations.

In monitoring water quality, various modes of contaminant exposure cages, flow-through tanks, and sediment exposure can be used as known in the art. Generally, the fish will be held in aluminum cages anchored to cement blocks submersed within specific bodies of water.

The above-described zebrafish are useful to monitor water quality. As such, the present invention provides for a method for using transgenic zebrafish with an easily assessable reporter gene under the control of pollutant-inducible DNA response elements. Transgenic zebrafish, carrying pollution-inducible response elements, are placed in the water to be tested, and the contaminants become bioconcentrated in the tissues of the fish thereby activating specific response elements, which up-regulate the bacterial lux reporter genes. Generally, the fish are then removed from the test water and placed immediately in a luminometer cuvette and incubated with luciferin. Luciferin is rapidly taken up into the tissues of the fish, oxidized by luciferase, and light is produced. The luminescence is proportional to the environmental concentration of the pollutant (to which the fish had been exposed), which drives the expression of the bacterial lux gene by means of the various DNA motifs. The luminescence is quantitated in the luminometer. In each response element-containing construct, the expression of the bacterial lux gene is activated by a specific class of polluting chemicals, allowing for differential identification of pollutants in a complex mixture.

In another embodiment, this disclosure provides a method of measuring contaminants in water, which includes exposing the transgenic zebrafish to a water sample to be tested for a time sufficient to allow contaminants within the water sample to become bioconcentrated within the zebrafish; exposing the transgenic zebrafish to conditions permitting expression of the reporter gene; and detecting the expression of the reporter gene. In some examples the methods include quantitating the detected expression by correlating to known standards and thereby detecting the quantity of contaminants in the water sample. In some examples, the reference standard is an aquatic source containing a known contaminant concentration. In another embodiment, the transgene is made up of multiple copies of the response element. In yet another embodiment, the transgene contains more than one type of response element. In yet another embodiment, the transgene contains more than two types of response element. In yet another embodiment, the transgene contains two or more copies each of more than one type of response element. In yet another embodiment, the transgene contains additional promoters or enhancers.

In another embodiment, the transgenic zebrafish is exposed to a water sample to be tested continually wherein the zebrafish is removed from the water sample repeatedly at selected intervals exposed to conditions permitting expression of the reporter gene and detected for reporter gene expression wherein such repeated exposures and detecting of expression is effective to track a time course of contaminant levels. Generally, the contaminant to be detected is selected from the group consisting of polyaromatic hydrocarbons, electrophilic oxidants heavy metals, endocrines, and retinoids. Preferably, the contaminant to be detected is selected from the group consisting of 2,3,7,8-tetrachlorodibenzo-p-dioxin, dioxin, polychlorinated biphenyls, quinones, mercury, copper, nickel, cadmium, zinc, estrogens, retinoic acid and 9-cis-retinoic acid.

Generally, the transgenic zebrafish are exposed to a water sample to be tested for a time sufficient to allow contaminants become bioconcentrated within the zebrafish. The exposure time is generally at least one minute. Preferably at least 2 minutes, more preferably at least one hour, more preferably at least 12 hours, more preferably at least 24 hours, more preferably at least one week, and more preferably at least two weeks. When the transgenic zebrafish is to remain exposed to the sample water for a longer duration in order to take multiple readings and create a time plot of contaminant levels, the total exposure time is generally at least at least 24 hours, more preferably is a time period chosen to be at least one week, at least two weeks, at least four weeks, at least eight weeks, at least 12 weeks, at least 24 weeks and at least 52 weeks.

The following examples are provided to illustrate certain particular features and/or embodiments. These examples should not be construed to limit the disclosure to the particular features or embodiments described.

### EXAMPLES

### Example 1

### Autonomous Bioluminescent Production in a Mammalian Cellular Background

### Materials and Methods

Strain Maintenance and Growth:

Escherichia coli cells were routinely grown in Luria Bertani (LB) broth with continuous shaking (200 rpm) at 37° C. When required, kanamycin or ampicillin was used at final concentrations of 40 and 100 μg/ml, respectfully, for selection of plasmid containing cells. Mammalian cell lines were propagated in Eagle's modified essential medium (EMEM) supplemented with 10% fetal bovine serum, 0.01 mM non-essential amino acids, and 0.01 mM sodium pyruvate. Cell growth was carried out at 37° C. in a 5% CO₂ environment and cells were passaged every 3-4 d upon reaching 80% confluence. Neomycin and/or zeocin were used for selection of transfected cells at concentrations of 500 μg/ml and 200 μg/ml, respectfully, as determined by kill curve analysis, for each antibiotic.

Codon Optimization of the Bacterial Bioluminescence Genes:

Codon usage patterns in the luxCDE genes for P. luminescens and the flavin reductase gene (frp) from V. harveyi were compared to the highest 10% of expressed genes as represented in GenBank. Silent mutations at the DNA level that would alter native codon usage were plotted to more closely mimic the preferred mammalian codons while maintaining 100% amino acid identity with the bacterial protein sequences. When multiple codons were preferred in equal or near equal frequencies by mammalian genes, the codon for the optimized sequence was randomly selected from the available options. These optimized sequences were submitted and synthesized de novo by GeneArt and returned as synthetic DNA constructs inserted into unique KpnI and Sad restriction sites in pPCR-Script vectors (GeneArt). Codon-optimized versions of each gene were compared to their wild-type counterpart for predicted translational efficiency using the freely available GENSCAN software at http://genes.mit.edu. All sequences were deposited to GenBank under the following accession numbers GQ850533 (codon-optimized luxC), GQ850534 (codon-optimized luxD), GQ850535 (codon optimized luxE), and GQ850536 (codon-optimizedfrp).

Vector Construction:

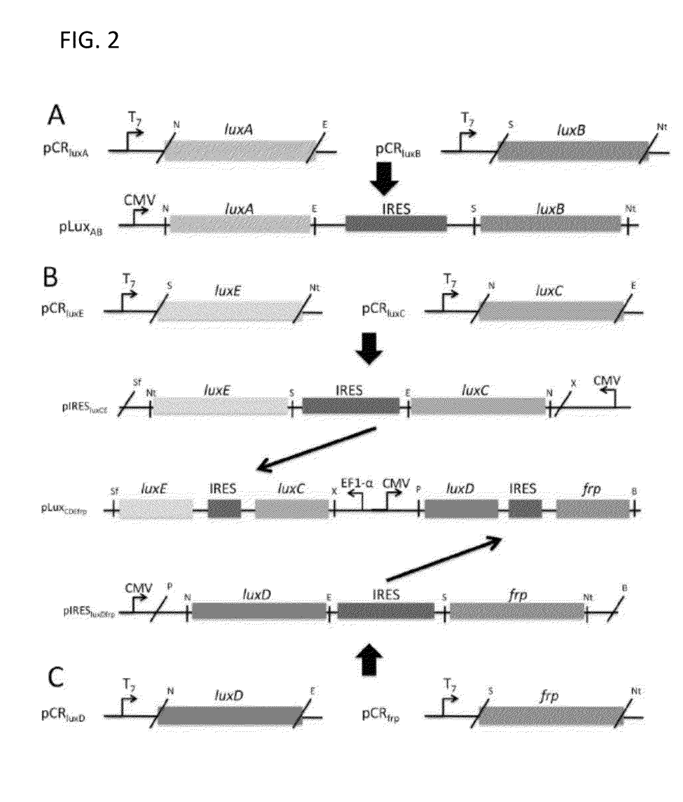

Previously described P. luminescens luxA and luxB genes partially codon-optimized for expression in human cell lines were obtained as a bicistronic operon in a pIRES vector (Clontech) and designated pLuxAB. This vector includes an internal ribosomal entry site (IRES) for increased translation of downstream gene insert. The remaining P. luminescens genes (luxC, luxD, luxE) and the flavin reductase gene (frp) were used in either their wild-type (wt) or codon optimized (co) states. coluxC was cloned into multiple cloning site (MCS) A of the pIRES vector using the unique NheI and EcoRI restriction sites (FIG. 2A-C). The coluxE gene was then inserted into MCS B using the unique SalI and NotI restriction sites. This entire pLuxCDEfrp:WT.

Mammalian Cell Transfection:

Transfection was carried out in six-well Falcon tissue culture plates (Thermo-Fisher). HEK293 cells stably expressing the pLux_{AB} vector were passaged into each well at a concentration of approximately 1×10⁵ cells/well and grown to 90-95% confluence in complete medium as described above. pLux_{CDEfrp:CO} and pLux_{CDEfrp:WT} plasmid vectors were purified from 100 ml overnight cultures of E. coli using the Wizard Purefection plasmid purification system (Promega). On the day of transfection, cell medium was removed and replaced and vector DNA was introduced using Lipofectamine 2000 (Invitrogen).

Selection of Stable Bioluminescent Cell Lines:

Twenty-four hours post-transfection, the medium was removed and replaced with complete medium supplemented with the appropriate antibiotic. Selection of successfully transfected clones was performed by refreshing selective medium every 4-5 days until all untransfected cells had died. At this time, colonies of transfected cells were removed by scraping, transferred to individual 25 cm² cell culture flasks, and grown in complete medium supplemented with the appropriate antibiotics.

Protein Extraction:

Total protein was extracted from co-transfected pLux_{CDEfrp:CO}/pLux_{AB} and pLux_{CDEfrp:WT}/pLux_{AB} cell lines using a freeze/thaw procedure. Cells were first grown to confluence in 75 cm² tissue culture flasks, then mechanically detached and resuspended in 10 ml of PBS. Following collection, cells were washed twice in 10 ml volumes of PBS, pelleted and resuspended in 1 ml PBS. These 1 ml aliquots of cells were subjected to three rounds of freezing in liquid nitrogen for 30 sec, followed by thawing in a 37° C. water bath for 3 min. The resulting cell debris was pelleted by centrifugation at 14,000 g for 10 min and the supernatant containing the soluble protein fraction was retained for analysis.

Bioluminescent Detection:

Bioluminescence was measured using an FB 14 luminometer (Zylux) with a 1 sec integration time. To prepare the sample for in vitro bioluminescent measurement, 400 μl of the isolated protein extract was combined with 500 μl of either oxidoreductase supplemented light assay solution containing 0.1 mM NAD(P)H, 4 μM FMN, 0.2% (w/v) BSA and 1 U of oxidoreductase protein isolated from V. fischeri (Roche), or oxidoreductase deficient light assay solution (distilled water substituted for the 1 U of oxidoreductase protein). Following the initial bioluminescent reading, samples were amended with 0.002% (w/v) n-decanal and the readings were continued to determine if additional aldehyde could increase light output. All bioluminescent signals were normalized to total protein concentration as determined by BCA protein assay (Pierce) and reported as relative light units (RLU)/mg total protein. All sample runs included processing of cell extracts from HEK293 cells stably transfected with pLux_{AB} as a control for light expression upon amendment. To prepare cells for in vivo bioluminescent measurement, the total cell contents of a 75 cm² tissue culture flask were resuspended in 1 ml of Dulbecco's Modified Eagle Medium (DMEM) without phenol red supplemented with 10% fetal bovine serum, 0.01 mM non-essential amino acids, and 0.01 mM sodium pyruvate. A 15 μl aliquot of cells was removed and counted using a hemocytometer to allow all values to be normalized to viable cell counts. The remainder was used directly for bioluminescent measurement using the FB14 luminometer with a 1 sec integration time.

Growth Curve Analysis:

Cells were harvested during exponential growth from a 75 cm² tissue culture flask and split into four 25 cm² tissue culture flasks at ˜5 #104 cells/cm². At 24 h intervals, the cells were detached from the flasks by mechanical agitation and resuspended in 3 ml phosphate buffered saline (PBS). A 15 μl aliquot was removed and diluted into an equal volume of trypan blue. Cells were counted using a hemocytometer and the average of 4 counts was used to determine the total viable cell number.

Determination of Minimum Detectable Cell Number in Culture:

Actively growing HEK293 cells expressing pLux_{CDEfrp}:CO/pLux_{AB} were trypsinized and harvested from 75 cm2 tissue culture flasks and counted using a hemocytometer. Using a 24-well tissue culture plate, groups of approximately either 500,000, 250,000, 100,000, 50,000, 40,000, 30,000, 20,000, 10,000, 5,000, 2,000, or 1,000 cells were plated in each of three wells in 1 ml of DMEM without phenol red supplemented with 10% fetal bovine serum, 0.01 mM nonessential amino acids, and 0.01 mM sodium pyruvate. As a negative control, three wells were supplemented with 1 ml of media without cells to observe background. Average radiance in photons/sec/cm²/sr was determined in the IVIS Lumina using a 10 min integration time 15 h after plating.

Correlation of Cell Population Size and Bioluminescent Output:

To establish the relationship of cell number to bioluminescent flux, the average radiance values from cells producing a visible light signal under the conditions above were correlated to cell number.

Cellular Harvesting and Preparation for Injection:

Actively growing HEK293 cells expressing pLux_{CDEfrp:CO}/pLux_{AB} were trypsinized and harvested from 225 cm² tissue culture flasks and counted using a hemocytometer. Using the average of two counts with the hemocytometer, cells were resuspended at approximately 5×10⁶ cells/100 μl PBS in a 1.5 ml tube (Eppendorf). Cells were maintained at 37° C. in a water bath until required for injection.

Whole Animal Bioluminescent Imaging:

Five week old nu/nu (nude) mice were anesthetized via isoflurane inhalation until unconscious. Subjects were then subcutaneously injected with ˜5×10⁶ HEK293 cells co-transfected with pLux_{CDEfrp:CO}/pLux_{AB} in a 100 μl volume of PBS. An equal number of HEK293 cells (˜5×10⁶) containing only pLux_{AB} were injected as a negative control in the same volume. The subject was imaged immediately following the injections and average radiance was determined over integration times of 1 to 10 min at intervals over a 30 min period.

Determination of Minimum Detectable Cell Number Following Subcutaneous Injection:

Six week old nude mice were anesthetized via isoflurane inhalation until unconscious and then injected with decreasing numbers of HEK293 cells expressing pLux_{CDEfrp:CO}/pLux_{AB}. In a preliminary experiment, animals were subcutaneously injected at 4 separate locations with 5 million, 2.5 million, 1 million, and 500,000 cells, each in a volume of 100 μl PBS. The subject was imaged for 10 min following injection of the final group of cells. Minimum detectable cell numbers were further delineated in a second round of injections in a fresh mouse model using cell concentrations of 500,000, 250,000, 50,000, and 25,000 cells in 100 μl PBS and identically imaged.

### Results

Benchtop Bioluminescent Detection from Stably Transfected HEK293 Cells:

To determine and compare the bioluminescent output kinetics of HEK293 cells containing the luxCDEfrp genes in either their wild-type (pLux_{CDEfrp:WT}) or codon-optimized (pLux_{CDEfrp:CO}) form, cells were propagated under identical conditions, harvested, and resuspended directly in a cuvette for measurement of bioluminescence against a standard photomultiplier tube interface. Cells containing pLux_{CDEfrp:CO}/pLux_{AB} showed an average bioluminescent production 12-fold greater than background in the presence of untransfected control cells and 9-fold greater than the bioluminescent production of their wild-type counterparts (Table 1). The superior bioluminescent production by cells containing pLux_{CDEfrp:CO}/PLux_{AB} validates the disclosed dual plasmid, bicistronic, codon optimized expression strategy and substantiates our hypothesis that the full bacterial lux cassette can be designed for functional autonomous expression in a mammalian cell line.

TABLE 1
Bioluminescent production from unsupplemented HEK293 cells
expressing P. luminescens lux genes.

| | Bioluminescent Detection |
|---|---|
| Cell Line | (RLU/sec) |
| Cell Free Media | 745 (±63) |
| Untransfected | 655 (±44) |
| HEK293 Cells | |
| HEK293 + | 884 (±44) |
| pLux_{AB} + | |
| pLux_{CDEfrp}:WT | |
| HEK293 + | 7600 (±1241) |
| pLux_{AB} + | |
| pLux_{CDEfrp}:CO | |

Growth Curve Analysis:

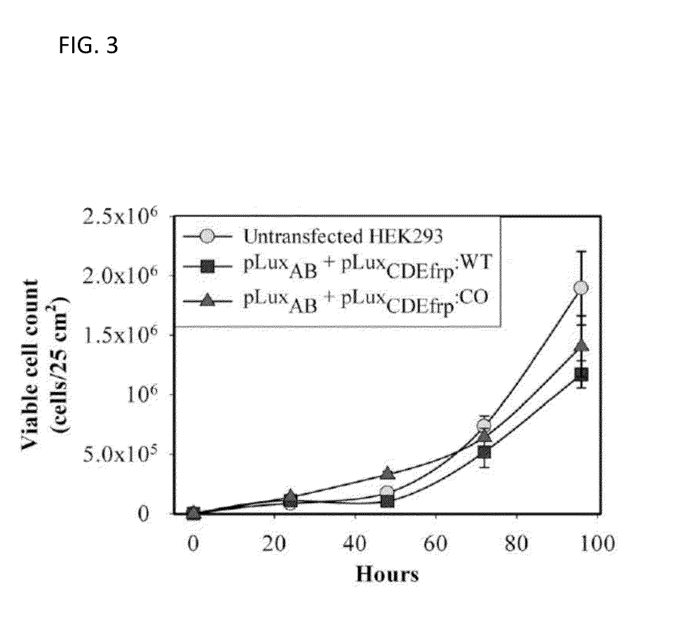

To determine if the maintenance and expression of full complements of lux genes was detrimental to cellular growth rates in HEK293 cells, the rates of growth among wild-type, pLux_{CDEfrp:CO}, and pLux_{CDEfrp:WT} containing cells was monitored over the course of a normal passage cycle. It was hypothesized that any adverse effects from production of aldehyde or increased presence of FMNH₂ resulting from the expression of the pLux_{CDEfrp} plasmid would result in a slowed growth rate relative to the wild-type HEK293 cell line. No significant difference in the rates of growth was observed among any of the cell lines tested (FIG. 3), suggesting that any adverse effects resulting from expression of the luxCDEfrp genes are minimal in regards to cellular growth and metabolism.

Bioluminescent Detection from Cell Culture:

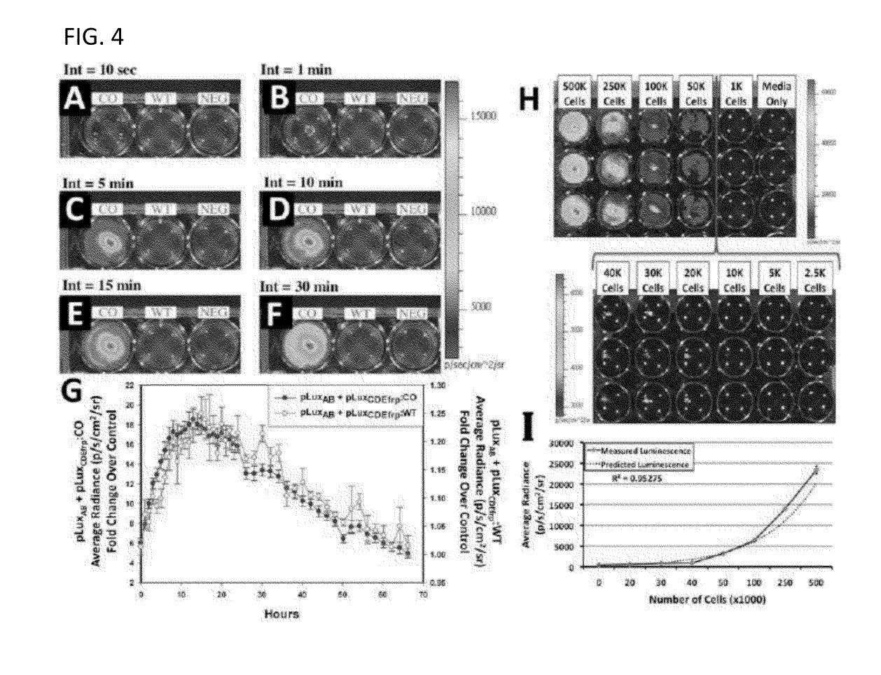

For a lux-based system to function as a reporter in whole animal BLI, the resulting signal must be detectable using commercially available equipment designed for this purpose and be easily distinguishable from background light emissions. To determine if this was the case in HEK293 cells expresing full lux cassettes, approximately equal numbers of cells containing either codon optimized or wild-type lux genes were plated in 24-well tissue culture plates and compared with untransfected cells as a negative control for background. The bioluminescent signal from cells co-transfected with codon-optimized luxCDEfrp was differentially detectable from background using a 10 sec integration time (FIG. 4A) and increased in magnitude with no appreciable increase in background up to integration times of 30 min (FIG. 4B-4F). To determine the maximal duration of the bioluminescent signal during constitutive expression under experimental conditions, approximately equal numbers of HEK293 cells in either their untransfected state or containing pLuxAB co-transfected with either pLux_{CDEfrp:WT} or pLux_{CDEfrp:CO} were continually monitored for bioluminescence production (FIG. 4G) in an IVIS Lumina imaging system using a stage temperature of 37° C. to mimic as closely as possible their normal growth conditions. Cells containing the lux cassette genes demonstrated bioluminescent output over an approximate three-day period without any exogenous input. Peak bioluminescent output was achieved between 12 and 13 h for both the codon-optimized and wild-type containing cell lines, however, following peak bioluminescent output a slow decrease in bioluminescent production over time was observed. This decrease is presumably due to a combination of the inability to reliably regulate the air temperature, CO₂ levels, and humidity in the imaging system, and the continued depletion of nutrients from the media during the normal process of cellular growth and metabolism. While the bioluminescent output of cells containing pLux_{CDEfrp:WT}/pLux_{AB} was of a lesser magnitude than that of their codon-optimized counterparts over this time period, their bioluminescent expression profiles were similar under the same conditions, suggesting that the codon-optimization process had not significantly altered the function of the lux proteins in vivo.

To be useful as an optical reporter, cells expressing bioluminescence must be detectable over a dynamic population range. To determine the minimum detectable cell number, HEK293 cells containing pLux_{CDEfrp:CO}/pLux_{AB} at concentrations ranging from 1,000 to 500,000 cells were plated in triplicate in equal volumes of media over a constant surface area and imaged over a 10 min integration time. The minimum number of cells reliably detected above background was approximately 20,000 although some visible signal was detected at approximately 10,000 cells in at least one case (FIG. 4H).

A major advantage imparted by the use of bioluminescent or fluorescent tagged reporter cells is that they allow an investigator to approximately quantify the population size of those cells noninvasively in a living host. For this approximation to be made using a lux-based system, it must be demonstrated that the bioluminescent flux of the cell population correlates tightly with the overall population size. To determine if this is the case in HEK293 cells constitutively expressing codon-optimized bacterial luciferase genes, the average radiance of cells producing a visibly detectable bioluminescent signal was determined over cell concentrations ranging from 500,000 to 1,000 cells. The average radiance closely correlated with the number of cells present (R²=0.95275) over all visibly detectable cell numbers tested (FIG. 4I).

Bioluminescent Detection from a Small Animal Model System:

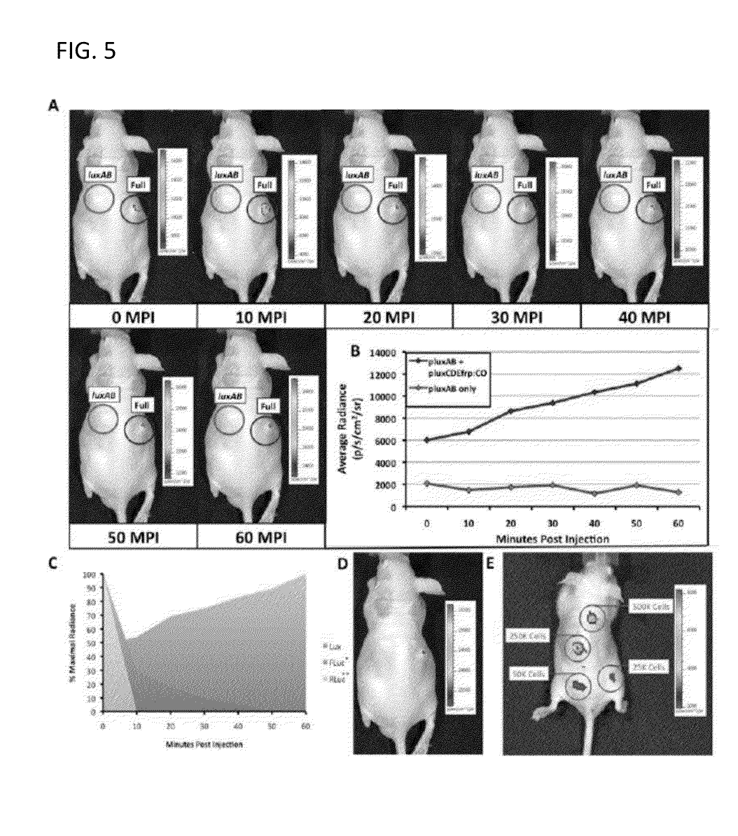

Although lux has been previously used in whole animal BLI (Contag et al. 1995), this is the first demonstration of its functionality outside of a bacterial host. Bacteria-free expression of this genetic system assures that the results seen are directly related to the object of study, and are not artifacts of a host-pathogen interaction stemming from the previously required bacterial infection. To demonstrate this functionality, 5 week old nude mice were subcutaneously injected with HEK293 cells co-transfected with pLux_{CDEfrp:CO}/pLux_{AB} or pLux_{AB} alone and imaged. Cells containing only pLux_{AB} were injected as a negative control to determine if the substrates supplied by the luxCDEfrp genes in the pLux_{CDEfrp} plasmid were capable of being scavenged from endogenously available stocks within the host in the presence of the luciferase dimer formed by the products of the luxAB genes on the pLux_{AB} plasmid. Bioluminescent signal emission from injected pLux_{CDEfrp:CO}/pLux_{AB} HEK293 cell lines was detectable immediately (<10 sec) following injection (FIG. 5A), mirroring the results of subcutaneous tumor mimic bioluminescence from firefly luciferase (FLuc)-tagged and Renilla luciferase (RLuc)-tagged cells following intravenous (IV) injection of their D-luciferin or coelenterazine substrates, respectfully. Following injection, the lux signal increased slowly in intensity over the full 60 min course of the assay (FIG. 5B). This is in contrast to FLuc-based bioluminescent signals that exhibit a steady decline, over the same period following IV injection of D-luciferin to a level ˜20% of their initial intensity (Inoue, Kiryu et al. 2009). RLuc bioluminescence is even more temporally limited and subsides within 5 min following IV injection of coelenterazine (Bhaumik and Gambhir 2002) (FIG. 5C). In contrast, the lux bioluminescent signal remained detectable 60 min after injection using integration times as low as 30 sec (FIG. 5D). Conversely, FLuc signals are asymptotically approaching their minimum and RLuc signals have become fully attenuated by 30 min, thus making imaging at all but the shortest post-injection incubation times impossible (FIG. 5C). It is important to note that the duration of the bioluminescent signal in FLuc containing systems can be extended by using a subcutaneous or intraperitoneal injection of luciferin, however, each injection route also produces a different bioluminescent emission profile over time. Because they forgo the addition of exogenous substrates to trigger bioluminescence, lux-based systems are not subject to these effects. The lack of a signal after injection of cells expressing only pLuxAB at any of the time points sampled confirms that the luciferase dimer alone is not capable of producing unintended bioluminescence above the background levels of light detection by scavenging endogenously available substrates. These results demonstrate the utility of the lux system in providing bioluminescent data on relatively prolonged time scales without the potentially error-inducing requirement of disturbing the experimental environment to invasively inject additional luciferin substrate.

Having illustrated the ability to reliably detect at least 20,000 cells in a tissue culture setting, the minimum detectable number of cells in small animal models remained to be determined. The detection of bioluminescent cells following subcutaneous injection is more difficult than detection in a culture setting due to the increased presence of chromophoric material leading to higher absorption of emitted photons as they must travel through more tissue to reach the detector. Subcutaneous injections of decreasing numbers of cells into a nude mouse model revealed that the introduction of at least 25,000 cells was capable of producing a detectable signal (FIG. 5E). As predicted from the correlation of cell number to bioluminescent flux, injection of higher cell concentrations produced larger bioluminescent signals over identical integration times.

### Discussion

Development of the lux cassette into a functional and autonomous mammalian bioluminescent system provides researchers a unique new tool that allows for real-time monitoring of bioluminescence from whole animals or cell cultures without exogenous substrate addition or cell lysis. The first step in the creation of this reporter was the functional demonstration of the luciferase heterodimer formed by the luxAB genes. This set the stage for the use of lux in eukaryotic cells as a non-autonomous reporter system via the addition of aldehyde. Since that time, the production of aldehyde has been demonstrated in S. cerevisiae, leading to the development of the first eukaryotic lux-based autonomous reporter system. As disclosed herein, it has been demonstrated for the first time that expression of codon-optimized forms of the luxCDE genes from P. luminescens and the frp gene from V. harveyi are capable of producing sufficient levels of the aldehyde and FMNH₂ substrates required to drive light production autonomously in mammalian cells. It is further demonstrated that these bioluminescent cells can be applied in whole animal BLI without the need for substrate addition.

While the addition of luxCDEfrp to cells containing luxAB demonstrates light emission at a level 12-fold greater than background (Table 1), it remained to be determined if the associated increase in aldehyde production would be cytotoxic, as had been demonstrated in luxAB containing S. cerevisiae and Caenorhabditis elegans cells. If this scenario was determined to be true, the increased presence of aldehyde may therefore cause those cells capable of most efficiently producing aldehyde to inhibit their own growth, mimicking the effects of antibiotic selection and causing them to be out-competed in culture by cells expressing lower levels of aldehyde production. This investigation revealed no significant variation among the growth rates of untransfected HEK293 cells or those expressing either pLux_{CDEfrp:WT}/pLux_{AB} or pLux_{CDEfrp:CO}/pLux_{AB} at levels capable of supporting continuous bioluminescent production (FIG. 3). These cells are necessarily producing the required aldehyde substrate as demonstrated by their constitutive bioluminescent production, but do not show a detectable difference in their rate of growth when compared to cells that are grown under identical conditions but without the luxCDE genes required for the production and maintenance of the aldehyde substrate.

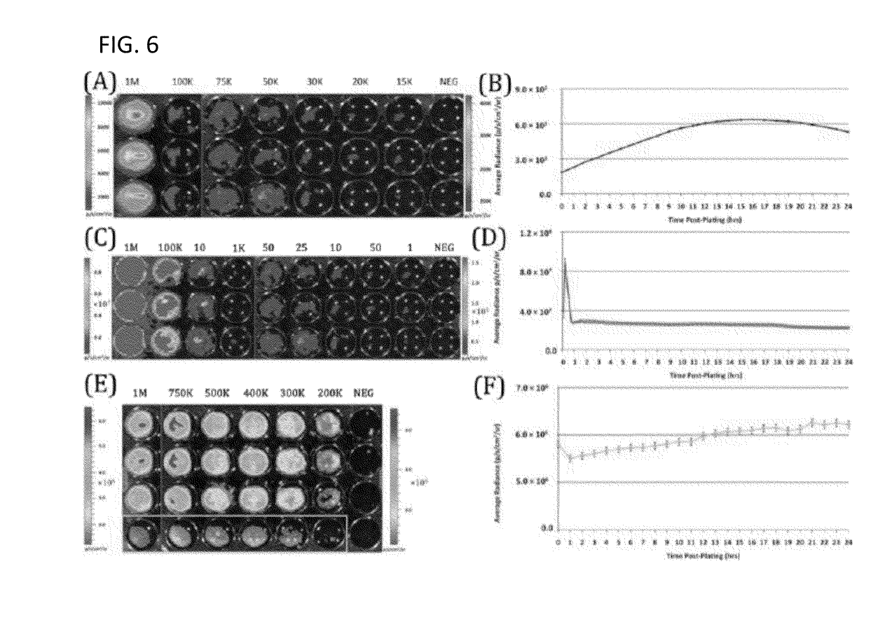

When these codon-optimized lux containing HEK293 cells were used in cell culture, concentrations of approximately 20,000 cells were reliably detected in 1 ml of media immediately using a 10 min integration time (FIG. 4H). Increasing cell numbers in the same volume and area correlated with measured levels of bioluminescence emission, allowing one to predict the total cell number in a given sample from the measured average radiance (FIG. 6I) and permitting non-invasive estimation of target size based on bioluminescent measurements.

When the same bioluminescent cell lines were applied in whole animal BLI, the low levels of detectable background signal and deficit of endogenous bioluminescent production associated with mammalian cells enabled lux-based bioluminescence to remain detectable. This sensitivity was demonstrated both in cell culture and under subcutaneous whole animal BLI conditions where very little light is produced due to attenuation of the bioluminescent signal by absorption from endogenous chromophores. It has been demonstrated here that cells co-transfected with the codon-optimized luxCDEfrp genes can produce a lasting signal that can be amplified over integration times as long as 30 min with little to no background to interfere with signal acquisition (FIG. 4F) in a cell culture setting. However, it is important to note that the bioluminescent signal from this reaction is produced at 490 nm. This is relatively blue-shifted as compared to the Luc-based bioluminescent probes that display their peak luminescent signal at 560 nm. The shorter wavelength of the lux-based signal has a greater chance of becoming attenuated within the tissue and therefore may not be as easily detected if it is used in deeper tissue applications (such as intraperitoneal or intraorganeller injections), and may require longer integration times to achieve the same level of detection as a longer wavelength reporter would when injected subcutaneously.

The use of cells expressing bacterial luciferase genes as a probe for whole animal BLI solves many of the problems associated with the currently available luciferase-based imaging systems. Previous work with lux genes isolated from P. luminescens has demonstrated that the luciferase is thermostable at the 37° C. temperature required for mammalian imaging experiments. This prevents the associated loss of signal associated with the short half-life of the firefly luciferase, which has been shown to be thermolabile at 37° C. in its native state. In addition, the autonomous nature of bioluminescent production associated with the lux system circumvents continuous re-injection of the test animal with an exogenous luciferin substrate. This simultaneously reduces the amount of invasive injections required for imaging experiments, eliminates the detection of artificial results stemming from any non-specific biological reactions with the luciferin compound being administered, and negates the inability to compare otherwise similar experiments due to differential bioluminescent production kinetics based on dissimilar routes of substrate injection. Thus, the bacterial luciferase offers a more specific, longer lasting, and more humane luciferase based reporter system than the currently available alternatives.

While mammalian-adapted bacterial luciferase gene expression has some notable disadvantages such as requisite introduction of multiple gene sequences and bioluminescent production at a wavelength that is relatively highly absorbed in mammalian tissues, it remains easily detectable using currently available imaging technology and offers several important advantages over the currently available reporter systems for prolonged expression without the cost or disturbance to the system associated with substrate administration. It is shown here that expression of the luxCDEfrp genes in mammalian cells can produce the requisite co-substrates for bioluminescent production and that codon optimization of these genes improves their performance—leading to an overall increase in light production as compared to their wild-type counterparts. When co-expressed with the luxAB genes responsible for formation of the luciferase heterodimer, aldehyde production occurs at a level capable of inducing autonomous light production, but not of high enough concentration to be adversely cytotoxic. When cells containing full complements of lux genes are enlisted as probes in whole animal BLI, they are easily detectable when introduced at levels comparable to cells expressing other currently employed target luciferase genes and allow for facile differentiation from background over prolonged integration times at 37° C., making them ideal reporter systems for cell culture, subcutaneous, or other low absorption environments that require prolonged, real-time monitoring without disruption.

### Example 2

### Comparison of Mammalian-Adapted Bacterial Bioluminescence with Firefly Luciferase Bioluminescence and Fluorescence from the Green Fluorescent Protein

As disclosed herein, it has been demonstrated that autonomous bioluminescent production from a mammalian cell line expressing human-optimized (ho) bacterial luciferase (lux) cassette genes can be used as a target for cell culture and small animal bioluminescent imaging (BLI). In this example, the bioluminescent expression of a mammalian HEK293 cell line transfected with the holux genes is compared with the bioluminescent expression of the same cell line expressing a commercially available, ho-firefly luciferase gene (luc) and the fluorescent expression of a commercially available, improved green fluorescent protein (GFP). The luc and gfp genes are two of the most widely known and used reporter genes for optical imaging and therefore provide excellent points of comparison for determining if holux expression would be beneficial in a given experiment.

### Materials and Methods

Strain Maintenance and Growth:

Escherichia coli cells were routinely grown in Luria Bertani (LB) broth with continuous shaking (200 rpm) at 37° C. When required, kanamycin or ampicillin was used at final concentrations of 40 and 100 μg/ml, respectfully, for selection of plasmid containing cells. Mammalian cell lines were propagated in Eagle's modified essential medium (EMEM) supplemented with 10% fetal bovine serum, 0.01 mM non-essential amino acids, and 0.01 mM sodium pyruvate. Cell growth was carried out at 37° C. in a 5% CO₂ environment and cells were passaged every 3-4 d upon reaching 80% confluence. Neomycin and/or zeocin were used for selection of transfected cells at concentrations of 500 μg/ml and 200 μg/ml, respectfully, as determined by kill curve analysis, for each antibiotic.

Transfection:

Transfection was carried out in six-well Falcon tissue culture plates (Thermo-Fisher). HEK293 cells were passaged into each well at a concentration of ˜4×10⁵ cells/well in complete medium the day before transfection. Plasmid vectors were purified from 100 ml overnight cultures of E. coli using the Wizard Purefection plasmid purification system (Promega). On the day of transfection, cell medium was removed and replaced and vector DNA was introduced using Lipofectamine 2000 (Invitrogen).

Selection of Stable Cell Lines:

Twenty-four h post-transfection, the medium was removed and replaced with complete medium supplemented with the appropriate antibiotic. Selection of successfully transfected clones was performed by refreshing selective medium every 4-5 d until all untransfected cells had died. At this time, colonies of transfected cells were removed by scraping, transferred to individual 25 cm² cell culture flasks, and grown in complete medium supplemented with the appropriate antibiotics.

Screening of Firefly Luciferase Containing HEK293 Cell Lines:

Following stable selection with antibiotics, cells containing the luc2 gene were tested to preferentially isolate lines producing the greatest luminescent signal following addition of D-luciferin. Cells were passaged from individual 25 cm² culture flasks (Corning) to individual wells of an optically clear 24-well culture 78 plate (Costar) and grown until confluence (1-2 d). Upon reaching confluence cells were lysed by application of 1× lysis buffer (Stratagene). Plates were then transferred to a Synergy 2 microplate reader (BioTek) and luminescence was measured following addition of 100 μl substrate-assay buffer (Stratagene) using an 8 sec delay and 10 sec integration time. The cell line producing the greatest luminescent output during testing was selected and maintained for experimental use.

Screening of GFP Containing HEK293 Cell Lines:

Following stable selection with antibiotics, cells containing the gfp gene were tested to preferentially isolate the line producing the greatest fluorescent output signal upon excitation. Cells were passaged from individual 25 cm² culture flasks (Corning) into black 24-well culture flasks (Costar) in a 2 ml volume of PBS. Immediately following passage cells were assayed for fluorescent production in a Wallac 1420 Multilabel counter (Perkin Elmer) using an excitation wavelength of 485 nm and a 510 nm emission filter. Cells lines producing the highest levels of fluorescence under these conditions were then subjected to a second round of testing. In the second stage, cells stably expressing GFP were grown to confluence in 25 cm² culture flasks (Corning) and harvested by trypsination. Cell counts were obtained as the average of two counts using a hemocytometer and cells were plated into black 24-well culture plates (Costar) at a concentration of ˜4×10⁶ cells/well in a 1 ml volume of PBS. Cells were then assayed for fluoresce in an IVIS Lumina in vivo imaging system (Caliper Life Sciences) using the GFP filter set and a 1 sec integration time. The cell line displaying the highest fluorescent emission signal under these conditions was selected and maintained for experimental use.

Dynamics of Lux Bioluminescent Production Over Time:

Actively growing HEK293 cells expressing pLux_{CDEfrp:CO}/pLux_{AB} (holux) were trypsinized and harvested from cm² tissue culture flasks (Corning) and viable cell counts were determined as the average of two counts using a hemocytometer. Approximately 1×10⁶ cells per well were plated in each of three wells in opaque 24-well tissue culture plates (Costar) in DMEM without phenol red and supplemented with 10% fetal bovine serum, 0.01 mM nonessential amino acids, and 0.01 mM sodium pyruvate. Along with the transfected cells, an equal number of untransfected HEK293 cells were plated to determine background luminescent detection levels. Photon counts were recorded using an IVIS Lumina in vivo imaging system and analyzed with Living Image 3.0 software (Caliper Life Sciences). The change in light output over time was determined in photons (p)/sec/cm²/steridian (sr) for each well using integration times of 10 min and reported as the average of three runs with the standard error of the mean.

To determine the minimum detectable population size, serial dilutions of cells ranging from ˜1×10⁶ cells per well to ˜100 cells per well were plated in each of three wells in opaque 24-well tissue culture plates in DMEM without phenol red and supplemented with 10% fetal bovine serum, 0.01 mM nonessential amino acids, and 0.01 mM sodium pyruvate. Along with the transfected cells, an equal number of untransfected HEK293 cells were plated to determine background luminescent detection levels. Photon counts were recorded using an IVIS Lumina in vivo imaging system and analyzed with Living Image 3.0 software (Caliper Life Sciences). Average radiance for all population sizes was determined in photons (p)/sec/cm²/steridian (sr) for each well using an integration time of 10 min 17 h post-plating, as this was shown to be the period of maximum bioluminescent production as determined by tracking the dynamics of bioluminescent output as described above. Following initial analysis, a more specific minimum detectable population size was determined by performing a second assay using cell concentrations ranging between the lowest detectable number of the initial assay and the highest undetectable number of cells plated and comparing the average radiance of each population to the level of background light detected over cell-free medium. For all measurements, statistical differences were determined by using Student's t tests with a p value cutoff of p=0.05.

Dynamics of LUC Bioluminescent Production Over Time:

Actively growing HEK293 cells expressing pGL4.50[luc2/CMV/Hygro] (Luc) were trypsinized and harvested from 75 cm2 tissue culture flasks (Corning) and viable cell counts were determined as the average of two counts using a hemocytometer. Approximately 1×10⁶ cells per well were plated in each of three wells in opaque 24-well tissue culture plates (Costar) in DMEM without phenol red and supplemented with 10% fetal bovine serum, 0.01 mM nonessential amino acids, and 0.01 mM sodium pyruvate. Along with the transfected cells, an equal number of untransfected HEK293 cells were plated to determine background luminescent detection levels. Immediately prior to imaging, all wells were spiked with 0.07 mg D-luciferin/ml (Caliper Life Sciences). Photon counts were then recorded using an IVIS Lumina in vivo imaging system and analyzed with Living Image 3.0 software (Caliper Life Sciences). The change in light output over time was determined in photons (p)/sec/cm2/steridian (sr) for each well using integration times of 10 sec and reported as the average of three runs with the standard error of the mean.

To determine the minimum detectable population size, serial dilutions of cells ranging from ˜1×10⁶ cells per well to ˜400 cells per well were plated in each of three wells in opaque 24-well tissue culture plates in DMEM without phenol red and supplemented with 10% fetal bovine serum, 0.01 mM nonessential amino acids, and 0.01 mM sodium pyruvate. Along with the transfected cells, an equal number of untransfected HEK293 cells were plated to determine background luminescent detection levels. Immediately prior to imaging, all wells were spiked with 0.07 mg D-luciferin/ml (Caliper Life Sciences). Photon counts were then recorded using an IVIS Lumina in vivo imaging system and analyzed with Living Image 3.0 software (Caliper Life Sciences). Average radiance for all population sizes was determined in photons (p)/sec/cm²/steridian (sr) for each well using an integration time of 10 sec immediately following the addition of luciferin, as this was shown to be the point of maximal bioluminescent output as determined by tracking the change in bioluminescent production dynamics as described above. Following initial analysis, a more specific minimum detectable population size was determined by performing a second assay using cell concentrations ranging between the lowest detectable number of the initial assay and the highest undetectable number of cells plated and comparing the average radiance of each population to the level of background light detected over cell free medium. For all measurements, statistical differences were determined by using Student's t tests with a p value cutoff of p=0.05.

Dynamics of GFP Bioluminescent Production Over Time:

Actively growing HEK293 cells expressing pcDNA3.1-CT-GFP (GFP) were trypsinized and harvested from 75 cm² tissue culture flasks (Corning) and 83 viable cell counts were determined as the average of two counts using a hemocytometer. Approximately 1×10⁶ cells per well were plated in each of three wells in opaque 24-well tissue culture plates (Costar) in either DMEM without phenol red and supplemented with 10% fetal bovine serum, 0.01 mM non-essential amino acids, and 0.01 mM sodium pyruvate or in PBS. Regardless of the assay medium, an equal number of untransfected HEK293 cells were plated to determine background fluorescent production levels upon addition of the excitation signal. Cells were stimulated in an IVIS Lumina in vivo imaging system using the supplied GFP excitation filter and photon counts were recorded using the supplied GFP emission filter. The resulting photon counts were analyzed with Living Image 3.0 software (Caliper Life Sciences). The change in fluorescent output over time was determined in photons (p)/sec/cm²/steridian (sr) for each well using integration times of 1 sec and reported as the average of three runs with the standard error of the mean.

To determine the minimum detectable population size, serial dilutions of cells ranging from ˜1×10⁶ cells per well to ˜100 cells per well were plated in each of three wells in opaque 24-well tissue culture plates in either DMEM without phenol red and supplemented with 10% fetal bovine serum, 0.01 mM non-essential amino acids, and 0.01 mM sodium pyruvate or in PBS. Regardless of the assay medium, an equal number of untransfected HEK293 cells were plated to determine background luminescent detection levels. Twenty-four h post plating, cells were stimulated in an IVIS Lumina in vivo imaging system using the supplied GFP excitation filter and photon counts were recorded using a 1 sec integration time with the supplied GFP emission filter. Twenty-four h post plating was chosen as this was determined to be the point of highest fluorescent radiance as determined by tracking the change in fluorescent production over time as described above. The resulting photon counts were analyzed with Living Image 3.0 software (Caliper Life Sciences) and recorded as photons (p)/sec/cm²/steridian (sr) for each well. All reported values represent the average of three runs with the standard error of the mean. For all measurements, statistical differences were determined by using Student's t tests with a p value cutoff of p=0.05.

Bioluminescent Measurement of Bacterial Luciferase Expressing HEK293 Cells in a Small Animal Model System

Preparation of Cells for Injection:

Actively growing HEK293 cells expressing pLux_{CDEfrp:CO}/pLux_{AB} (lux) were trypsinized and harvested from 225 cm² tissue culture flasks and counted using a hemocytometer. Using the average of two counts with the hemocytometer, cells were resuspended at approximately 5×10⁶ cells/100 μl PBS in a 1.5 ml tube (Eppendorf) for subcutaneous injection or at approximately 1×10⁷ cells/100 μl PBS in a 1.5 ml tube (Eppendorf) for intraperitoneal injection. Following resuspension, cells were maintained at 37° C. in a water bath until required for injection.

Subcutaneous Injection:

Five week old nu/nu (nude) mice (NCRNU-M, Taconic Farms Inc.) were anesthetized via isoflurane inhalation until unconscious. Subcutaneous injections of ˜5×10⁶ HEK293 cells expressing pLux_{CDEfrp:CO}/pLux_{AB} (lux) in 100 μl volumes of PBS were performed in both the shoulder and hip of each subject (n=3) for a total of n=6 subcutaneous injections. Due to the of the lack of endogenous bioluminescent processes in mammalian tissue, and to control for changes in overall animal size and dispersion of reporter-tagged cells following injection, readings were gathered as total flux values and presented in photons (p)/second (s). All subjects were imaged immediately following the injections using 1 min integration times. Total flux from each injection site was determined by drawing regions of interest (ROI) of identical size over each location. Readings were recorded once every 10 min over a 60 min period to determine the change in flux over time. To determine the minimal detectable number of cells in vivo, a subject was subcutaneously injected at three locations—the scruff of the neck, the mid back, and hip—with the relevant range of cells as determined by the previously described minimum detectable cell number assays under culture conditions. All cell concentrations were injected in a 100 μl volume of PBS. The subject was then imaged using integration times of up to 10 min to determine if a luminescent signal could be detected above background at the injected concentrations of cells. For all measurements, statistical differences were determined by using Student's t tests with a p value cutoff of p=0.05.

Intraperitoneal Injection:

To measure bioluminescent flux following intraperitoneal injection of the lux cell line, five week old nu/nu (nude) mice (NCRNU-M, Taconic Farms Inc.) were anesthetized via isoflurane inhalation until unconscious and each subject (n=2) then received a single injection of ˜1×10⁷ HEK293 cells expressing pLuxCDEfrp:CO/pLuxAB (lux) in a 100 μl volume of PBS. All subjects were imaged immediately following the injections using 1 min integration times. Total flux from each injection site, measured as p/s, was determined by drawing regions of interest (ROI) of identical size over each location. Readings were recorded once every 10 min over a 60 min period in order to determine the change in flux over time.

Bioluminescent Measurement of Firefly Luciferase Expressing HEK293 Cells in a Small Animal Model System

Preparation of Cells for Injection:

Actively growing HEK293 cells expressing luc2 (Luc) were trypsinized and harvested from 225 cm² tissue culture flasks and counted using a hemocytometer. Using the average of two counts with the hemocytometer, cells were resuspended at approximately 5×10⁵ cells/100 μl PBS in a 1.5 ml tube (Eppendorf) for subcutaneous injection or at approximately 1×10⁶ cells/100 μl PBS in a 1.5 ml tube (Eppendorf) for intraperitoneal injection. Following resuspension, cells were maintained at 37° C. in a water bath until required for injection.

Subcutaneous Injection;

Five week old nu/nu (nude) mice (NCRNU-M, Taconic Farms Inc.) were anesthetized via isoflurane inhalation until unconscious. Subcutaneous injections of ˜5×10⁵ HEK293 cells expressing luc2 (Luc) in 100 μl volumes of PBS were performed in both the shoulder and hip of each subject (n=3) for a total of n=6 subcutaneous injections. Due to the of the lack of endogenous bioluminescent processes in mammalian tissue, and to control for changes in overall animal size and dispersion of reporter-tagged cells following injection, readings were gathered as total flux values and presented in photons (p)/second (s). Following subcutaneous injection of the reporter cells, each subject was subjected to intraperitoneal injection of 150 mg D-luciferin/kg and then imaged immediately using 1 sec integration times. Total flux from each injection site was determined by drawing regions of interest (ROI) of identical size over each location. Readings were recorded once every 10 min over a 60 min period to determine the change in flux over time. To determine the minimal detectable number of cells in vivo, a subject was subcutaneously injected at three locations—the scruff of the neck, the mid back, and hip—with the relevant range of cells as determined by the previously described minimum detectable cell number assays under culture conditions. All cell concentrations were injected in a 100 μl volume of PBS. Prior to imaging, the subject was intraperitoneally injected with 150 mg D-luciferin/kg. The subject was then imaged using integration times of up to 10 min to determine if a luminescent signal could be detected above background at the injected concentrations of cells. For all measurements, statistical differences were determined by using Student's t tests with a p value cutoff of p=0.05.

Intraperitoneal Injection:

To measure bioluminescent flux following intraperitoneal injection of the Luc cell line, five week old nu/nu (nude) mice (NCRNU-M, Taconic Farms Inc.) were anesthetized via isoflurane inhalation until unconscious and each subject (n=2) then received a single injection of ˜1×10⁷ HEK293 cells expressing luc2 (Luc) in a 100 μl volume of PBS. Following injection of the reporter cell line, all subjects were injected at the same location with 150 mg D-luciferin/kg and then imaged immediately following the injections using 10 sec integration times. Total flux from each injection site, measured as p/s, was determined by drawing regions of interest (ROI) of identical size over each location. Readings were recorded once every 10 min over a 60 min period in order to determine the change in flux over time.

Fluorescent Measurement of GFP Expressing HEK293 Cells in a Small Animal Model System

Preparation of Cells for Injection:

Actively growing HEK293 cells expressing gfp (GFP) were trypsinized and harvested from 225 cm² tissue culture flasks and counted using a hemocytometer. Using the average of two counts with the hemocytometer, cells were resuspended at approximately 5×10⁶ cells/100 μl PBS in a 1.5 ml tube (Eppendorf) for subcutaneous injection or at approximately 1×10⁷ cells/100 μl PBS in a 1.5 ml tube (Eppendorf) for intraperitoneal injection. Following resuspension, cells were maintained at 37° C. in a water bath until required for injection.

Subcutaneous Injection

Five week old nu/nu (nude) mice (NCRNU-M, Taconic Farms Inc.) were anesthetized via isoflurane inhalation until unconscious. Subcutaneous injections of ˜5×10⁶ HEK293 cells expressing gfp (GFP) in 100 μl volumes of PBS were performed in both the shoulder and hip of each subject (n=3) for a total of n=6 subcutaneous injections. Subjects were imaged immediately following injection with the reporter cell line using GFP excitation and emission filter with an integration time of 1 sec. Total flux from each injection site was determined by drawing regions of interest (ROI) of identical size over each location. Readings were recorded once every 10 min over a 60 min period to determine the change in flux over time. To determine the minimal detectable number of cells in vivo, a subject was subcutaneously injected at three locations—the scruff of the neck, the mid back, and hip—with the relevant range of cells as determined by the previously described minimum detectable cell number assays under culture conditions. All cell concentrations were injected in a 100 μl volume of PBS. The subject was then imaged using integration times ranging from 0.5 sec to 3 min to determine if a fluorescent signal could be detected above background at the injected concentrations of cells. For all measurements, statistical differences were determined by using Student's t tests with a p value cutoff of p=0.05.

Intraperitoneal Injection:

To measure fluorescent flux following intraperitoneal injection of the Luc cell line, five week old nu/nu (nude) mice (NCRNU-M, Taconic Farms Inc.) were anesthetized via isoflurane inhalation until unconscious and each subject (n=2) then received a single injection of ˜1×10⁷ HEK293 cells expressing gfp (GFP) in a 100 μl volume of PBS. Total flux from each injection site, measured as p/s, was determined by drawing regions of interest (ROI) of identical size over each location following a 1 sec integration under GFP excitation and emission filters in the IVIS Lumina in vivo imaging system (Caliper Life Sciences). Readings were recorded once every 10 min over a 60 min period in order to determine the change in flux over time.

### Results

Bioluminescent Measurement of Bacterial Luciferase Expressing HEK293 Cells in Culture

Minimum Detectable Population Size:

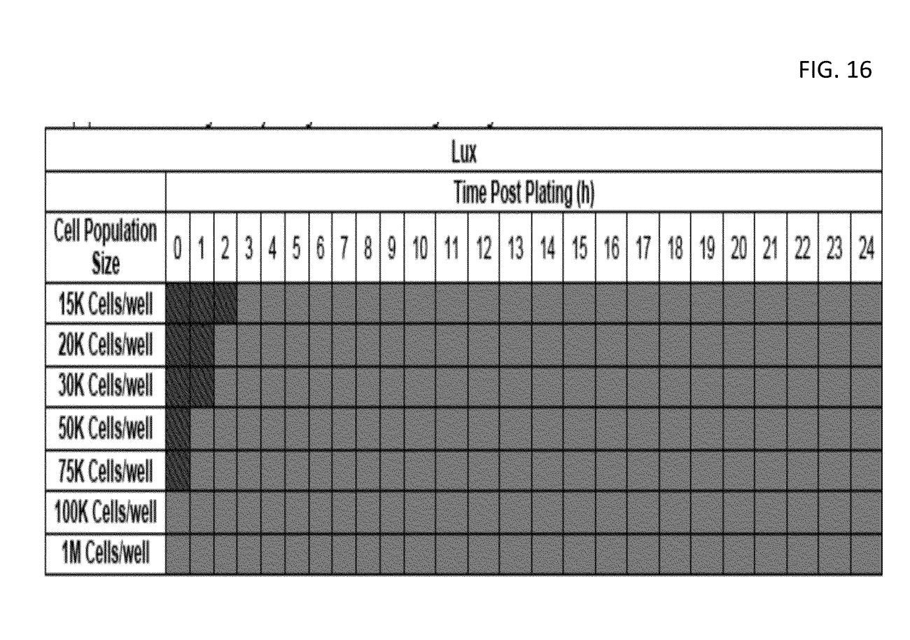

Cells expressing the holux cassette genes produced a visible light signal over a range from approximately 1×10⁶ cells/well to 1.5×10⁴ cells/well using a 10 min integration time (FIG. 6A). A detectable light signal was inconsistently observed at a concentration of ˜1×10⁴ cells/well, however this was determined not to be significantly distinguishable from background (p=0.72) (FIG. 6). In general, detection of lower cell populations as significantly different than background was more feasible at time points further from the initial plating (Table 2, FIG. 16).

Dynamics of Bioluminescent Production Over Time

The luminescent profile of holux expression demonstrated a consistent increase in average radiance from an initial post-plating value of 1,800 p/s/cm²/sr to a peak of 6,400 p/s/cm²/sr 16 h post-plating (FIG. 6B). Following peak bioluminescence, the cells expressed a slow decrease in average radiance over the remainder of the 24 hr assay, averaging a reduction of 135 (±16) p/s/cm²/sr per hr. Expression was consistent over the course of the assay with the standard error of the mean averaging 58 (±4) p/s/cm2/sr at each time point surveyed.

Bioluminescent Measurement of Firefly Luciferase Expressing HEK293 Cells in Culture

Dynamics of Bioluminescent Production Over Time:

The luminescent profile of the Luc expressing cells displayed a large initial intensity, with a peak average radiance of 8.9 (±0.4)×10⁷ p/s/cm2/sr 10 min following addition of 0.07 mg D-luciferin/ml. This level of radiance was not maintained, however, and had decreased to 3.0 (±0.3)×107 p/s/cm²/sr by 40 min post addition. The decrease in radiance occurred during the period 10 to 30 min post substrate addition, after which the signal remained steady (±9.3×10⁵ p/s/cm²/sr) for the remainder of the assay. Concurrent with the higher bioluminescent output of the Luc expressing cells compared to holux was a larger standard error. The average error over the course of the Luc luminescence assay was 2.9 (±0.6)×10⁶ p/s/cm2/sr (FIG. 6D).

Minimum Detectable Population Size:

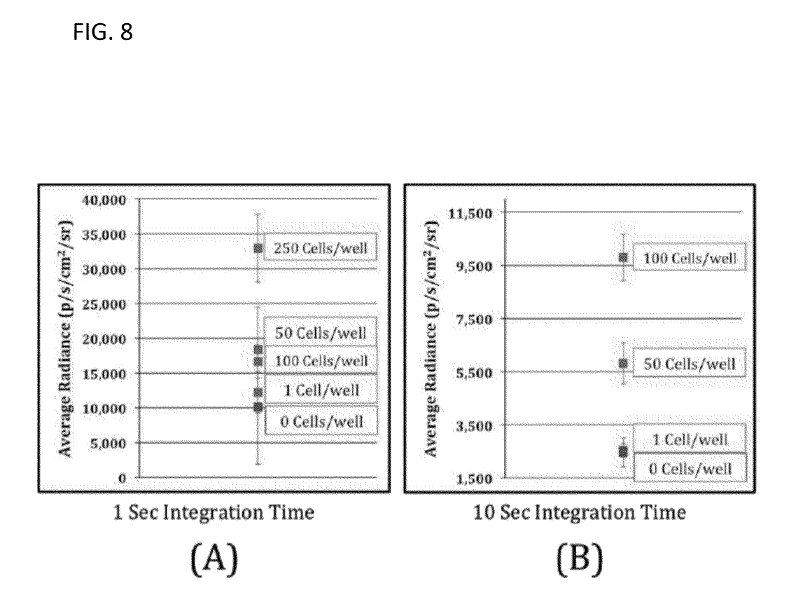

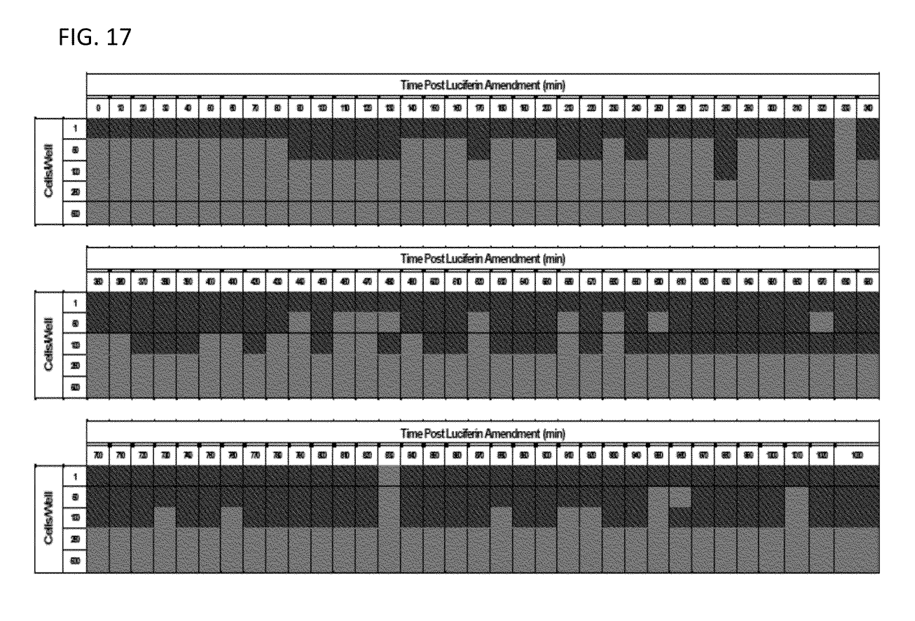

Cells expressing the human-optimized luc2 gene displayed a significantly greater average radiance (p<0.01) than those expressing the human-optimized lux genes and as a result were visible at lower concentrations. Luc expressing cells produced a visible signal over a range from ˜1×10⁶ cells/well down to 250 cells/well at an integration time of 1 sec (FIG. 6C and FIG. 8A). Although concentrations as low as 50 cells/well could be differentiated from background if the integration time was extended to 10 sec (FIG. 8B), this concentration of cells was not determined to be statistically greater than background light detection (p=0.65) while using the 1 sec integration time required to prevent saturation of the camera at the higher cell concentrations (FIG. 8A). Detection of the Luc-tagged cell populations showed the opposite trend of those expressing the holux genes and was generally easier to differentiate from background at time points closer to luciferin addition (Table 3, FIG. 17).

Fluorescent Measurement of GFP Expressing HEK293 Cells in Culture in Cell Culture Medium

When HEK293 cells expressing GFP were tested in DMEM without phenol red it was not possible to detect the fluorescent signal of the cells above the background level of fluorescent detection at any of the population sizes surveyed. In order to better differentiate the target signal from background, the integration time was lowered down to a minimum of 0.5 sec, however this had no effect on the ability to distinguish signal from noise. Based on these results it was concluded that the assay would be performed in PBS, as this media did not contain any serum proteins or compounds to contribute to the production of nonspecific background signal in the presence of the GFP excitation signal.

Dynamics of Bioluminescent Production Over Time:

Average radiance of HEK293 cells stably expressing GFP in a PBS as a medium increased slightly but not significantly (p=0.08) over the course of the assay from an initial value of 6.0 (±0.06)×10⁶ p/s/cm²/sr to a peak of 6.6 (±0.07)×10⁶ p/s/cm²/sr by 22 h after the initial plating. Over the full course of the assay the average radiance remained relatively steady at 6.2 (±0.05)×10⁶ p/s/cm²/sr with an average error of 7.3 (±0.3)×10⁴ p/s/cm²/sr (FIG. 6F).

Minimum Detectable Population Size:

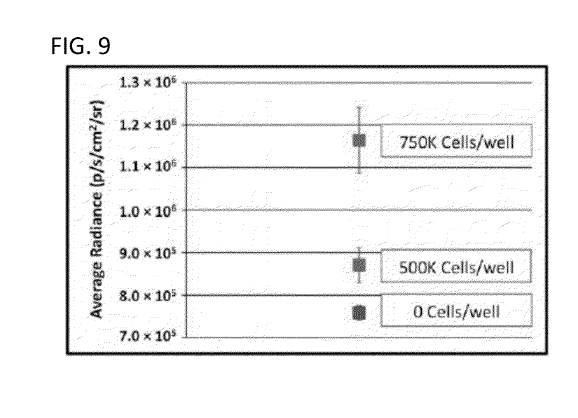

Fluorescent detection from GFP emission presented the least sensitive lower limits of detection for any of the three reporter systems tested when PBS was used as the assay medium. Under these conditions detection ranged from ˜1×10⁶ cells/well down to 5×10⁵ cells/well (FIG. 6E). Although wells of less than ˜5×10⁵ cells/well clearly show fluorescent signals, they were not significantly different from background following subtraction of background tissue autofluorescence (FIG. 9). Similar to the holux-expressing cells, detection ability increased for the smaller population sizes over the course of the assay (Table 4). A full comparison of the pertinent expression data for all three reporter systems is detailed in Table 5.

Bioluminescent Measurement of Bacterial Luciferase Expressing HEK293 Cells in a Small Animal Model System

Subcutaneous Injection:

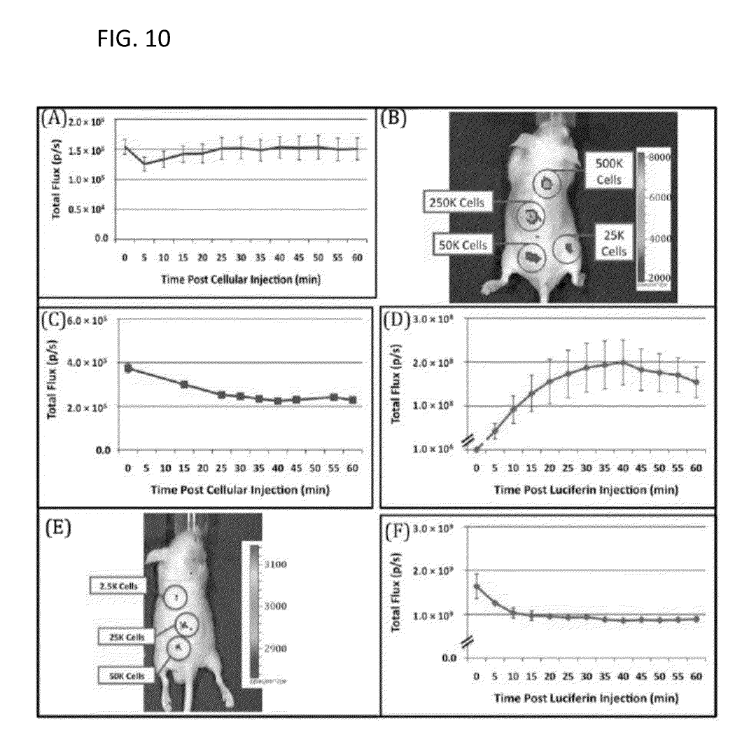

Average flux from subcutaneous injection of ˜5×10⁶ holux expressing cells was 1.5 (±0.2)×10⁵ p/s and remained relatively constant over the full course of the 60 min assay, displaying a minimum flux of 1.3 (±0.1)×10⁵ p/s and a maximum of 1.5 (±0.2)×10⁵ p/s. The standard errors of the readings were relatively low, averaging 1.6 (±0.3)×10⁴ p/s and therefore provide readings with increased resolution compared to the Luc reporter system. Over the full course of the assay, the bioluminescent profile remained relatively flat, displaying a range of 2.8×10⁴ p/s between the lowest and highest recorded values (FIG. 10A). To obtain a representative pseudocolor image during acquisition, integration times of 1 min were used, however, it was previously demonstrated that detection following subcutaneous injection of ˜5×10⁶ holux expressing cells is possible using ˜30 sec integration times. It was also demonstrated that following subcutaneous injection the lower level for detection was 25,000 cells when using increased integration times (−10 min) (FIG. 10B).

Intraperitoneal Injection:

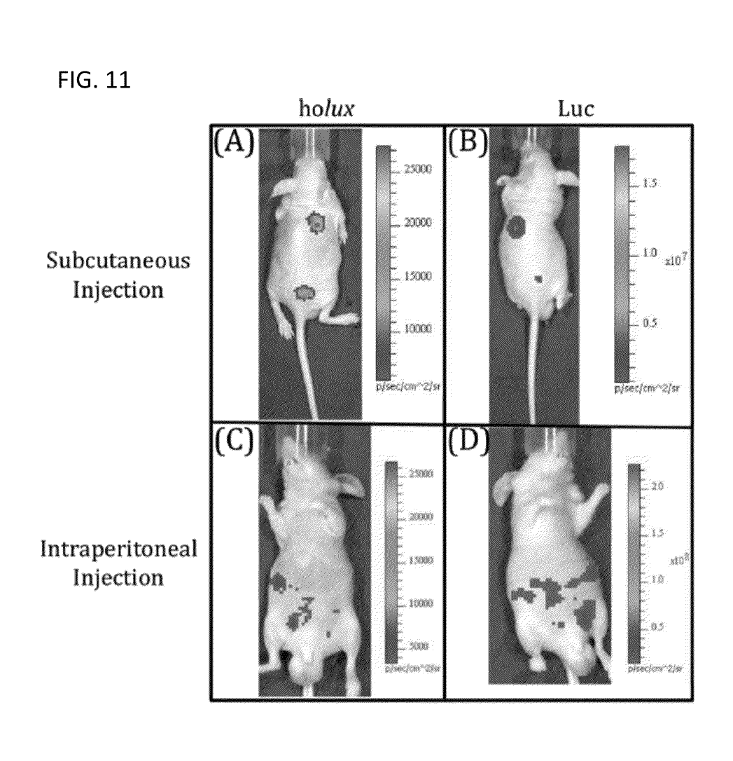

Intraperitoneal injections of ˜1×10⁷ holux expressing cells yielded a disparate bioluminescent profile from that of the subcutaneous injections. The largest total flux was measured immediately following injection at a rate of 3.6 (±0.2)×10⁵ p/s. Following this initial light output, the total flux continued to trend downward over the remainder of the assay (FIG. 10C). The greatest decrease, presumably from dispersion of the cells following injection, occurred during the first 15 min, during which the total flux decreased from the maxima to 2.4 (±0.2)×10⁵ p/s. After this time, the rate of bioluminescent production remained relatively flat, decreasing ˜67,000 p/s by the final time point of the 60 min assay. Due to the diffusion of cells within the intraperitoneal cavity following injection and the increased amount of scattering and absorption associated with intraperitoneal imaging, pseudocolor images obtained using a 60 sec integration time were not as well defined as those from the subcutaneous injections despite the injection of a higher number of cells (FIGS. 11A and C). The expression value differences (in p/s) that lead to these changes in pseudocolor representation are presented in Table 5.

Bioluminescent Measurement of Firefly Luciferase Expressing HEK293 Cells in a Small Animal Model System

Subcutaneous Injection;

Subcutaneous injection of ˜5×10⁵ Luc containing cells produced a bell curve of bioluminescent production. Immediately following intraperitoneal injection of 150 mg D-luciferin/kg the average total flux from each injection site was 1.0 (±0.2)×10⁶ p/s. Total flux then increased rapidly over the next 40 min to a maximum of 2.0 (±0.5)×10⁸ p/s before declining for the remainder of the 60 min assay. Along with the increased flux values were increased error ranges at each time point as compared to the ho/ux-expressing cell line. Standard error of each reading averaged 4.0 (±0.5)×10⁷ p/s (FIG. 10D). Visual detection of signal was never problematic, with a 1 sec integration providing ample exposure for facile visual representation of the subcutaneous injection site (FIG. 11B). With the system under the control of the CMV promoter, the minimum detectable cell number was determined to be 2,500 under subcutaneous imaging conditions (FIG. 10E).

Intraperitoneal Injection:

Intraperitoneal injections of ˜1×10⁶ Luc expressing cells produced a much different time dependent bioluminescent expression profile than that obtained following subcutaneous injections (compare FIG. 10F to FIG. 10D). The magnitude of bioluminescent flux notwithstanding, the time dependent bioluminescent profile following intraperitoneal injection of Luc expressing cells yielded a profile similar to that obtained following intraperitoneal injections of holux expressing cells (compare FIG. 10F to FIG. 10C). The highest total flux occurred immediately after intraperitoneal injection of 150 mg D-luciferin/kg at 1.6 (±0.3)×10⁹ p/s. The bioluminescent flux then quickly decreased to 1.0 (±0.1)×10⁹ p/s by 10 min post luciferin injection. For the remaining 50 min of the assay the total flux remained relatively constant, averaging 9.2 (±0.2)×10⁸ p/s. As with the holux-expressing cells, integration time had to be extended to obtain a representative visual image of the intraperitoneal injection site. Intraperitoneal injection of ˜1×10⁶ Luc-expressing cells, followed by immediate imaging post luciferin injection using a 10 sec integration time, produced a pseudocolor visual representation similar to the pseudocolor images obtained using a 60 sec integration time following injection of ˜1×10⁷ holux-expressing cells, but did not produce images that were as well defined as those following subcutaneous injection (FIGS. 11B and D). This is presumably due to the increases in absorbance and scattering associated with injection into the intraperitoneal cavity. A summary of the differences between Luc expression in vivo or in vitro following either a subcutaneous or intraperitoneal injection can be found in Table 5.

Fluorescent Measurement of GFP Expressing HEK293 Cells in a Small Animal Model System

Subcutaneous Injection:

Subcutaneous injections ranging from ˜1×10⁴ to ˜1×10⁷ GFP expressing cells failed to produce a detectable fluorescent signal when expressed in a nude mouse model. When regions of interest were drawn over the injection site of ˜1×10⁷ cells in a 100 μl volume of PBS, these locations did not produce significantly more fluorescent flux then was measured over background from a region of identical size distal from the injection site (p=0.739). The location of the injection did not have a statistically detectable effect on the strength of the resulting fluorescent signal, with injection into the shoulder or the rump resulting in similar levels of detection for equal numbers of injected cells (p=0.050).

Intraperitoneal Injection:

Similar to the results obtained following subcutaneous injection, intraperitoneal injection of GFP expressing cells at population sizes up to ˜1×10⁷ were not able to be detected at any time point during the 60 min course of the assay. Regions of interest of identical size drawn either over the injection site, or distal from the injection site at an area not expected to display fluorescent signal displayed similar levels of fluorescent flux across all surveyed time points (p=0.100).

### Discussion

There have been myriad demonstrations of the bioluminescent and fluorescent profiles obtained in culture or small animal imaging when employing the Luc or GFP proteins as targets. The variety and scope of published literature utilizing these, or versions of these, reporters is testament to their usefulness, as well as the expression strategies to which they can be adapted within the confines of a particular experimental design. To aid in the comparison of the three different systems under conditions that are as uniform and comparable as could be achieved, each was expressed in the same cellular background (HEK293) and placed under the control of identical cytomegalovirus (CMV) promoters. The use of identical promoters should encourage similar levels of expression when each construct is expressed in the HEK293 cell line. However, in the holux cell line, although luxAB is driven by the CMV promoter, the luxC and luxE genes are instead under the control of the human elongation factor 1″ (EF-1″) promoter. Because the previously demonstration of holux function was designed in this manner it was not subjected to any modification prior to expression in order to allow for consistent comparison with the previously published results. As expected, bioluminescence from the Luc system was detectable at lower cell concentrations and displayed a significantly larger total flux than holux containing cells in the mouse imaging experiments and its detection level was lower than both the lux and GFP reporters in the cell culture imaging scenarios. Under conditions where only small populations of Luc-expressing cells were assayed in cell culture as few as 50 Luc cells/well were visible (FIG. 6C and FIG. 8B) compared with a minimum of 15,000 cells/well for the holux system (FIG. 6A) and 500,000 cells/well in the GFP system when cells were imaged in PBS (FIG. 6E and FIG. 9). The need to use PBS as a liquid medium to detect lower GFP-expressing cell numbers due to the autofluorescence from the cell culture medium represents a crucial problem with using fluorescent systems for prolonged cell culture imaging. The lack of medium components such as serum and nutrients required for low-level fluorescent detection does not promote continued cellular growth, thereby preventing potential autonomous fluorescent monitoring without regular medium changes. The inclusion of these compounds can prevent this, but increases the minimum detectable cell number beyond 1 million cells/well, and therefore could not be detected under our imaging conditions.

Another approach to overcome the poor sensitivity of GFP in culture is to use an alternate cell line capable of more efficiently expressing the reporter. It has previously been demonstrated that GFP expression under the control of the CMV promoter in the MCF-7 breast cancer cell line is capable of being detected at lower numbers of GFP expressing cells/well. however, these experiments were conducted in wells of significantly smaller surface area (0.32 cm² as compared to 1.9 cm²) than used in these experiments. When the results from both experiments are normalized to media volume, this corresponds to a lower detection level of ˜250 cells/μl using MCF-7 cells compared to ˜500 cells/μl when expressed in HEK293 cells. The results presented herein demonstrate, however, that the use of bioluminescence rather than fluorescence overcomes this problem completely; however, there is a large difference in the bioluminescent output levels and imaging strategies between the holux and Luc systems. The holux system has the advantage of not requiring addition of a substrate to elicit bioluminescent production, therefore allowing for completely autonomous bioluminescent readings that should routinely correlate with cell number, regardless of time. The disadvantage of the holux system is that it is significantly less efficient than the Luc system. While the average radiance of ˜1×10⁶ holux cells had a peak value of 6,400 p/s/cm²/sr, this is comparable to the peak average radiance of only ˜100 HEK-Luc cells/well (although this number of cells/well cannot be reliably detected following the initial bioluminescent burst following substrate amendment as shown in Table 2). Therefore, detection of small numbers of cells in culture is best suited to a Luc based reporter system, especially if the production of light is only to be monitored over short time periods. However, if working with larger cell populations, the use of a holux-based reporter system gives the benefit of continuous bioluminescent output, and is not dependent on the addition of luciferin to the cell culture medium. Regardless of which reporter system is employed, the use of a bioluminescent system (either holux or Luc) has the advantage of low background detection when compared with the use of a fluorescent system such as GFP in a medium-based cell culture setting. When applied to small animal imaging, the same general benefits for each reporter system are reiterated. The major disadvantage of working with GFP or alternate fluorescent reporter systems in an animal model is the relatively high level of background fluorescence resulting from excitation of endogenous chromophoric material within the subject tissue. The use of a bioluminescent reporter helps to overcome this disadvantage due to the low levels of background autoluminescence in mammalian tissues. While Luc based systems have most often been utilized for small animal imaging, the holux system provides a distinct advantage for near-surface target visualization. Although not as bright as the Luc system (total flux averaged 1.5 (±0.2)×10⁵ p/s for a subcutaneous injection of ˜5×10⁶ HEK293 holux cells vs. an overall average total flux of 1.4 (±0.2)×10⁸ p/s for a subcutaneous injection of ˜5×10⁵ Luc cells) the bioluminescent profile of the holux-containing cells was relatively flat over the full course of the assay, while the bioluminescent profile of the Luc containing cells varied greatly following substrate injection. In addition, the act of luciferin supplementation encompasses its own set of concerns. It has been well documented that the bioluminescent profile can be altered depending on the route of substrate administration for Luc-based, with each route having different uptake rates throughout the body. Also of concern, the process of substrate injection allows for the introduction of error due to differences in the efficiency of each injection and/or the possibility of potential injection failure (i.e. injection into the bowel during intraperitoneal administration). Any changes in the quality of the luciferin over time during multiple injections as well as the possible introduction of tissue damage that can prohibit further injections are also of concern. For large-scale experiments, the cost of luciferin must also be taken into consideration, as it is an expensive substrate. Therefore, the use of a holux-based reporter is more simplistic and economical and may provide more reliable results if relatively large numbers of cells are being imaged close to the surface of the subject. The inability to detect injections of GFP-expressing HEK293 cells at concentrations up to ˜1×10⁷ is in line with what has been previously reported in the literature. It has been previously demonstrated that HEK293 cells expressing GFP were not detectable until 7 d post injection when population sizes of 1×10⁶ cells were used for injection. With the doubling time of HEK293 cells reported to be 34 h, these cells should have reached a population size of ˜1×10⁷ by ˜5 days, two full days prior to when they were first reported to be detectable. When GFP is expressed in other cell lines, however, the time until detection can change. It has been reported that injection of ˜1×10⁷ GFP expressing MCF-7 cells was possible 1 d following injection, however, no information was given as to the detection ability immediately following injection. Although it may have been possible to elicit a detectable fluorescent signal by injection a higher concentration of GFP expressing cells, injection size was limited to ˜1×10⁷ cells because this was the largest injection size commonly reported in the literature. While previous reports have suggested that detection of a single cell expressing the luc reporter gene is possible in the 4T1 mouse mammary tumor line, it is shown here that a minimum of 2,500 cells are required when the Luc system is expressed in HEK293 cells under the control of a CMV promoter (FIG. 10E). Despite the increase in cells required for detection under our expression conditions, this number was still well below that required for detection of the holux-expressing cells (FIG. 10B). The diminished performance of the holux cells compared to Luc-containing cells during both minimal detection level testing and intraperitoneal injection demonstrates that the associated benefits of the holux system are of little value if they cannot be easily detected under experimentally relevant imaging conditions. In cases where deep tissue imaging is required, the use of a Luc-based system can be advantageous despite the concerns associated with substrate addition, especially since its use in these types of experiments is widespread and well documented. Whether subcutaneous or intraperitoneal injection is chosen as the route of administration, it is important to realize that the decreased efficiency of the holux system as compared to the Luc system necessitates an increase in integration time to obtain similar detection levels (FIG. 11). The amount of time required for signal detection must therefore be considered in the context of a given experiment to determine if detection of the holux signal at a level similar to what a researcher may be accustomed to using a Luc-based system is acceptable. The greatest advantages of the new holux system, however, are the ability for researchers to integrate its use alongside other established fluorescent and bioluminescent systems and the ability to exploit the unique autonomous nature of lux bioluminescent expression with novel detection methods. Because the presence of fluorescently labeled cells would not be detected under bioluminescent imaging conditions (i.e. in the absence of an excitation signal), the location and size of bioluminescent signals could be determined and then differentiated from any fluorescent signals detected following administration of the excitation signal. In addition, the holux signal could be determined prior to substrate injection in conjunction with alternative bioluminescent reporter systems to sequentially determine the location and size of differentially labeled cell populations within a living host. Alternatively, the autonomous nature of lux bioluminescent expression could allow it to be paired with miniaturized integrated circuit microluminometers that could one day be implanted under the skin of an animal subject, allowing for real-time detection of signal without the need for external imaging equipment. This possibility opens the door for development of integrated biofeedback circuits that can autonomously monitor and subsequently react to numerous in vivo disease conditions. So while the introduction of a holux imaging target certainly does not displace the use of currently available fluorescent and bioluminescent imaging targets, it can overcome some of the shortcomings of these systems and integrates well with them as an additional tool for noninvasive imaging.

### Example 3

### Use of Mammalian-Adapted Bacterial Luciferase Genes as a Reporter System for Use in the Mammalian Cellular Background

For many years researchers have been using bacteria and simple eukaryotes such as yeast to serve as proxies for measuring the bioavailability of exposed chemicals to human cells. These simple models have distinct advantages of being easy to manipulate in the laboratory, inexpensive to maintain, and highly amenable to high throughput experimental design. However, as attractive as they might be, they are not completely representative of human derived cells. As such, there is always some amount of caution that must be taken when interpreting the data obtained using these models and relating it to human bioavailability. Oftentimes human derived cells cannot be used for bioavailability screening because of the lack of reporter systems allowing for real-time, autonomous reporting of the associated effects.

As disclosed herein, the use of human derived HEK293 cells stably transfected with the luxCDABEfrp genes of the bacterial bioluminescence cassette (lux) is investigated as a means of overcoming the limitations imposed by currently available mammalian cell based bioavailability detection methods. To investigate the utility of these cells to act as biosensors for the presence of a specific target chemical, a version of the lux gene cassette was created that regulated the expression of the luxC and luxE genes in response to doxycycline using the commercially available TET-On system (Clontech). The TET-On system represents a common method for evaluating the effectiveness of a reporter system as it has previously been shown to be effective for expression of a wide array of reporter genes across multiple mammalian cell lines and therefore allows for the facile comparison of reporter function with previously published models. It has previously been demonstrated that bioluminescence can be detected from small numbers of human cells expressing the lux genes, and that the bioluminescent flux can be correlated to overall cell population size. This makes the substrate-free, real-time bioluminescent response of a lux-expressing cell line an excellent platform for development into a mammalian-based reporter system designed to signal target compound detection, as well as allowing for it to be developed into a first-of-its-kind biosentinel for toxic chemical exposure. The latter is made possible because the persistence of the bioluminescent signal without excitation or addition of substrate makes it possible to measure changes in overall bioluminescent production as an indicator for changes in cellular growth and metabolism. These types of measurements would not be possible using other reporter systems due to the economic and logistical concerns related to constantly simulating the reporter protein in order to generate the continuous signal required for real-time monitoring. The ability to autonomously produce a bioluminescent signal in response to a specific compound of interest without the requirement of investigator intervention allows for the possibility of high throughput, on-line, remote detection systems that could significantly improve on the cost and efficiency of current detection methods. In addition, the ability to efficiently screen multiple compounds in parallel in order to evaluate their potential cytotoxic effects directly on human cells gives the lux reporter system an advantage over other fluorescent or bioluminescent reporter systems in the field of toxicology.

### Materials and Methods

Strain Maintenance and Growth:

Escherichia coli cells were routinely grown in Luria Bertani (LB) broth with continuous shaking (200 rpm) at 37° C. When required, kanamycin or ampicillin was used at final concentrations of 40 and 100 μg/ml, respectfully, for selection of plasmid containing cells. Mammalian cell lines were propagated in Eagle's modified essential medium (EMEM) supplemented with 10% fetal bovine serum, 0.01 mM non-essential amino acids, and 0.01 mM sodium pyruvate. Cell growth was carried out at 37° C. in a 5% CO2 environment and cells were passaged every 3-4 d upon reaching 80% confluence. Neomycin, hygromycin, and/or zeocin were used for selection of transfected cells at concentrations of 500 μg/ml, 100 μg/ml, or 50 μg/ml, respectfully, as determined by kill curve analysis, for each antibiotic.

Cloning of the Tetracycline Response Element

PCR Amplification:

Primers were designed to amplify a 349 bp region of the pTRE-Tight-BI plasmid (Clontech) containing both the tetracycline response element and its associated CMV promoter sequence. The TET forward primer was engineered to contain an NheI restriction site, while the TET reverse primer was engineered with an associated NotI restriction site. This allowed for the attainment of an altered PCR product that contained a 5′ NheI restriction site, followed by the tetracycline response element and CMV promoter, then a 3′ NotI restriction site. The resulting PCR product was then immediately TOPO cloned into the pCR4-TOPO vector (Invitrogen) to create pCR4-TET. This plasmid was used as the basis for allowing propagation and maintenance of the receptor fragment.

Introduction of the Tetracycline Response Element into pLuxCDEfrp:

The tetracycline response element and its associated CMV promoter were removed from pCR4-TET using the NheI and NotI restriction sites. In parallel with this reaction, the EF1-α promoter was removed from pLuxCDEfrp using the same restriction sites.

Following restriction digest, both reactions were purified by gel electrophoresis. The ˜350 bp band representing the tetracycline response element and the CMV promoter were extracted from the lane containing the pCR4-TET digestion and the ˜8.9 kb band representing the pLux_{CDEfrp} plasmid with the EF 1-α promoter removed was extracted from the lane containing the pLUX_{CDEfrp} digestion. Both isolated fragments were purified using QIAquick Gel Extraction kits (Qiagen). The purified fragments were ligated together for 5 min at room temperature using T4 DNA polymerase (Promega) in LigaFast buffer (Promega) to create the plasmid pLux_{CDEfrp-TET}. The ligated plasmid DNA was then used directly for transformation. Chemically competent E. coli were inoculated with 2 μl of the pLux_{CDEfrp-TET} ligation product and selected by growth on LB medium containing 100 μg Ampicillin/ml. Successful uptake the pLux_{CDEfrp-TET} plasmid was confirmed by restriction digest and the success of the ligation reaction was confirmed by sequencing.

Transfection of pLuxAB and pLuxCDEfrp in HEK293 Cells:

Transfection was carried out in six-well Falcon tissue culture plates (Thermo-Fisher). The day prior to transfection, HEK293 cells were passaged into each well at a concentration of approximately 1×10⁵ cells/well and grown to 90-95% confluence in complete medium. pLux_{AB} and pLux_{CDEfrp-TET} plasmid vectors were purified from 100 ml overnight cultures of E. coli using the Wizard Purefection plasmid purification system (Promega). On the day of transfection, cell medium was removed and replaced and vector DNA mixed in a 1:1 ratio was introduced using Lipofectamine 2000 (Invitrogen).

Screening of Stably Transfected Reporter Cell Lines;

Twenty-four h post-transfection, the medium was removed and replaced with complete medium supplemented with the appropriate antibiotic. Selection of successfully transfected clones was performed by refreshing selective medium every 4-5 d until all untransfected cells had died. At this time, colonies of transfected cells were removed by scraping, transferred to individual 25 cm² cell culture flasks, and grown in complete medium supplemented with the appropriate antibiotics. To screen the resulting cell lines for the ability to regulate luxC transcription in response to doxycycline, relative reverse transcription PCR (rt-PCR) was used to determine the level of luxC mRNA present 24 h following the addition of 0, 10, or 100 ng doxycycline/ml to the complete growth medium. To this end, each isolated cell line was split into 4 25 cm² cell culture flasks upon reaching 80% confluence. Cells were then grown at 37° C. and 5% CO₂ until again reaching ˜80% confluence. At this point, one of the flasks was routinely passaged to maintain a stock of cells for future use. The remaining 3 flasks were spiked with 0, 10, or 100 ng doxycycline/ml and returned to the incubator. Twenty-four h post doxycycline addition, cells were harvested and total RNA was isolated using an RNeasy isolation kit (Qiagen). Isolated RNA was used directly for rt-PCR analysis, and cell lines displaying the greatest range in luxC transcription between 0 ng Doxycycline/ml treatment and either 10 ng or 100 ng doxycycline/ml treatment were selected for bioluminescent screening.

Bioluminescent Measurement in Response to Doxycycline:

Cell lines displaying the greatest upregulation of luxC gene transcription following treatment with 10 ng and 100 ng Doxycycline/ml were passaged into 75 cm² tissue culture flasks (Corning) and grown at 37° C. and 5% CO₂ until they reached 90% confluence. Cells were then harvested by trypsinization and cell number was determined as the average of two counts using a hemocytometer. Approximately 1×10⁶ cells per well were plated in triplicate in opaque 24-well tissue culture plates (Costar) in DMEM without phenol red and supplemented with 10% fetal bovine serum, 0.01 mM non-essential amino acids, and 0.01 mM sodium pyruvate. Immediately post plating, wells were spiked with either 0, 10, 100, or 500 ng Doxycycline/ml. Photon counts were then recorded using an IVIS Lumina in vivo imaging system and analyzed with Living Image 3.0 software (Caliper Life Sciences). The change in light output over time was determined in photons (p)/sec (s) for each well by averaging the photon output over integration times of 10 min and reported with the standard error of the mean.

Bioluminescent Measurement in Response to Toxic Chemical Exposure:

To determine the ability of constitutively bioluminescent HEK293 cells to function as a bioreporter for toxic chemical exposure, cells stably expressing pLux_{CDEfrp}/pLux_{AB} were exposed to increasing concentrations of the cytotoxic aldehyde n-decanal. HEK293 cells previously determined to be capable of continuous bioluminescent output from the expression of the pLux_{CDEfrp}/pLux_{AB} plasmids were passaged into 75 cm² tissue culture flasks (Corning) and grown at 37° C. and 5% CO² until they reached 90% confluence. Cells were then harvested by trypsinization and cell number was determined as the average of two counts using a hemocytometer. Approximately 1×10⁶ cells per well were plated in all wells in opaque 24-well tissue culture plates (Costar) in DMEM without phenol red and supplemented with 10% fetal bovine serum, 0.01 mM non-essential amino acids, and 0.01 mM sodium pyruvate. Triplicate wells were treated with serial dilutions of n-decanal ranging from 0.1% to 1×10⁻⁵%, with a control set receiving no n-decanal amendment to determine maximal bioluminescent expression. Photon counts were then recorded using an IVIS Lumina in vivo imaging system and analyzed with Living Image 3.0 software (Caliper Life Sciences). The change in light output over time was determined in photons (p)/sec/cm²/steridian (sr) for each well using integration times of 10 mM once every hour for 24 h and reported as the average of three runs with the standard error of the mean.

### Results

Regulation of luxC Transcription in Response to Doxycycline Dose:

Following antibiotic selection of HEK293 cells co-transfected with pLux_{AB}/pLux_{CDEfrp-TET}, 11 cell lines were established that were capable of growing efficiently under selective media conditions. Each of these lines was interrogated for the ability to up regulate luxC gene transcription following doxycycline amendment. The average CT value for luxC detection in negative control cells containing no doxycycline was 13.6 (±0.7) cycles. Following amendment with 10 ng doxycycline/ml, the average CT value dropped to 11.9 (±0.7), and following amendment with 100 ng doxycycline/ml the average dropped to 11.5 (±0.6). Of the eleven cell lines tested, only three showed significant (p<0.05) reductions in CT value at both 10 and 100 ng treatment levels as compared to the negative control, and also between the 10 and 100 ng treatment levels themselves (Table 6). The best performing cell line displayed a reduction of 3.5 cycles to reach CT upon amendment with 10 ng doxycycline/ml, which corresponds to approximately 11-fold increase in luxC transcription. Upon induction with 100 ng doxycycline/ml, this cell line displayed a reduction of 4.3 cycles to reach CT, an ˜20-fold increase in transcription, although this was a difference of less than one cycle to reach CT as compared to induction with 10 ng doxycycline/ml, it was determined to be a statistically significant increase in transcriptional level (p=0.027). This cell line was chosen for determination of bioluminescent output in response to doxycycline addition.

Bioluminescent Production in Response to Doxycycline Dose:

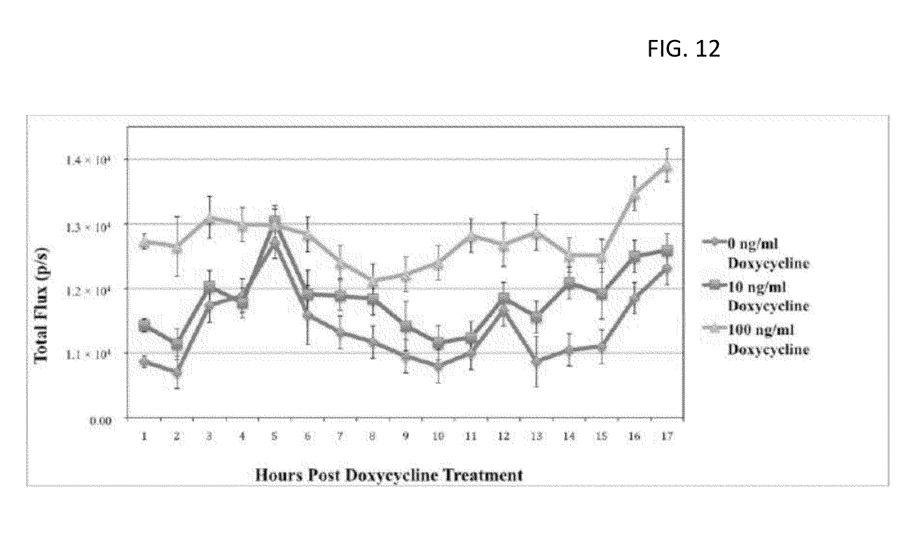

Using the cell line previously determined to have the most dynamic response to doxycycline treatment, cells were monitored to determine the magnitude and dynamics of bioluminescent production over a 24 h period following exposure to either 10 or 100 ng doxycycline/ml. Average background 8 bioluminescence detection from cells that did not receive doxycycline treatment was determined to be 11,000 (±205) p/s. Average total flux from cells treated with 10 ng doxycycline/ml was not increased significantly (p=0.06), although it did trend upwards to 11,500 (±200) p/s. Treatment with 100 ng doxycycline/ml, however, further increased the total flux to 12,500 (±200) p/s, a significant increase over both the negative (p=1.1×10⁻⁵) and over the 10 ng/ml treatment (p=9.2×10⁻⁴). These values remained relatively constant over the full course of the assay (FIG. 12), with ranges of only 2,010, 1,900, and 1,800 p/s for the negative control, 10 ng, and 100 ng doxycycline/ml treatments respectively following the initial evaluation at 1 h post treatment. It was possible to significantly distinguish the 100 ng/ml treatment level from background at all but the 5 h post treatment time point. While it was possible to significantly distinguish the 10 ng/ml treatment level at some of the time points, it was never consistently greater than that of the untreated control cells (Table 7). After it was discovered that bioluminescent production could be induced with treatment at 100 ng doxycycline/ml, but not at 10 ng doxycycline/ml, cells were treated with 500 ng doxycycline/ml to determine if higher treatment levels would be capable of inducing further increases in bioluminescent production. However, it was discovered that this treatment was not able to induce any further increase in bioluminescent output.

Use of Constitutively Bioluminescent Cells as Biosensors for n-Decanal Exposure:

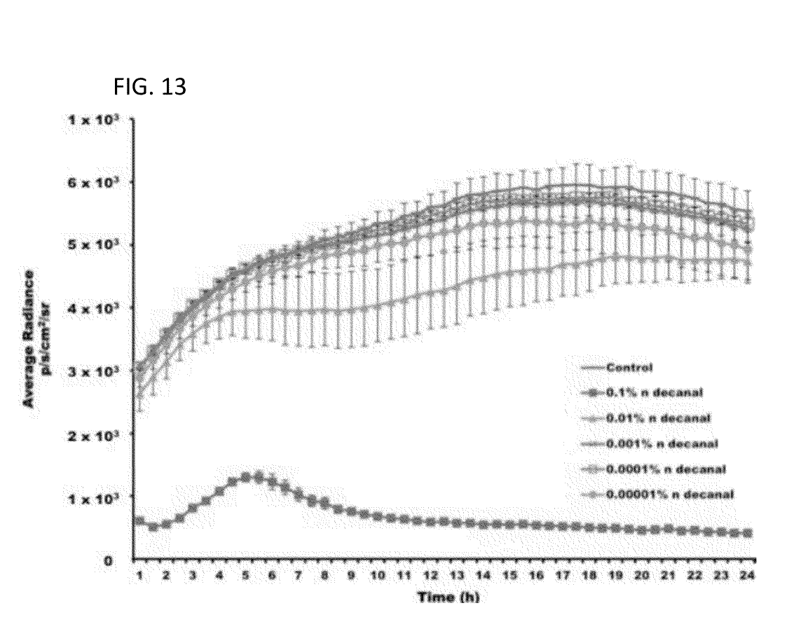

Constitutively bioluminescent HEK293 cells expressing pLux_{AB}/pLux_{CDEfrp} were exposed to increasing levels of the cytotoxic aldehyde n-decanal and the rate and magnitude of bioluminescent production was monitored to determine the bioavailability of this toxicant to a mammalian cell line. It was observed that treatment with 0.1% n-decanal reduced bioluminescent output immediately following addition, with all of the surveyed time points displaying significantly down regulated production of light (Table 8). Treatment with 0.01% n-decanal also had deleterious effects on bioluminescent production, however, due to the reduced concentration, these effects were not consistently observable until 4 h after addition (Table 8). The remaining treatment levels, while not capable of inducing increases in bioluminescence, could not be statistically differentiated from cells not receiving treatment (FIG. 13).

### Discussion

These findings represent the first use of autonomous bioluminescent production from a mammalian cell line being harnessed to directly detect the bioavailability of compounds of interest. The unique ability of the lux genes to produce a bioluminescent signal that is practically background free, without exogenous stimulation, opens the door for future development of high throughput, on-line monitoring systems. These types of screening systems have not previously been possible in the mammalian cellular background because of the photobleaching effects related to constant stimulation of fluorescent reporters or the prohibitively high cost of constant substrate profusion required for alternative bioluminescent reporters. With these barriers circumvented by the ability of the lux-expressing cells to produce light autonomously, there is now the potential to provide a facile method for screening large numbers of compounds simultaneously and directly relating the findings to human bioavailability. The ability of lux expression to function as a traditional bioreporter by modulating the expression of the luxC and luxE genes under control of a tetracycline responsive promoter has been demonstrated in these experiments. While this work has not demonstrated that this is in fact the most efficient method of regulation, it has validated that expression of the luxC and luxE genes can be used successfully to modulate bioluminescent expression at statistically significant levels (p<0.05). The luxC and luxE genes play a crucial role in the production and regeneration of the myristyl aldehyde substrate required by the lux luciferase enzyme in order to produce bioluminescence. The luxE gene encodes an acylprotein synthetase that activates an intermediate fatty acid compound to provide the energy required for its future reduction to an aldehyde. It is the luxC gene, which encodes a fatty acid reductase, that performs this reduction of the fatty acid precursor to form the aldehyde that ultimately takes part in the bioluminescent reaction. Previous work has demonstrated that constitutive expression of the luxA and luxB genes that form the actual luciferase enzyme is not cytotoxic and that expression of these genes alone is not sufficient to elicit bioluminescent production without the function of the remainder of the lux cassette genes. It has also been shown that high levels of aldehyde expression can be toxic in organisms exogenously expressing the lux genes. Taken together, these data indicate that regulation of aldehyde production within the mammalian cell will be the most efficient means for controlling bioluminescent expression while simultaneously maintaining efficient conditions for cellular growth and metabolism. While it may have been more efficient to control only a single gene (i.e. only luxC or luxE) rather than multiple genes to serve this purpose, it was necessary to simultaneously regulate two genes in order to properly mimic the polycistronic nature of the cassette within the mammalian host cells. Regulation of only one gene would have required re-engineering of the previously validated lux vectors, as well as the possible introduction of a third plasmid. This prospect would have been detrimental to transfection efficiency and significantly decreased the chances of successfully establishing a stable cell line expressing all three plasmid constructs.

Treatment of cells expressing the luxC and luxE genes under the control of the tetracycline responsive promoter with 100 ng doxycycline/ml was able to elicit a significant up regulation in bioluminescent output (p=1.1×10⁻⁵) following a relatively short incubation period of 1 h (FIG. 13). This time period is in line with previously published reports that have indicated the tetracycline responsive promoter is strong enough to produce detectable levels of its downstream gene product in as little as 30 min. It is not known why the bioluminescent levels of the control and 10 ng doxycycline/ml treated cells increased transiently during the 5 h time point (FIG. 12). This anomalous increase in flux from the control cell line represents the only surveyed time point where it was not possible to statistically differentiate the signal from cells treated with 100 ng doxycycline/ml from the negative control, however, it maintained the trend of non-statistically differential expression between the control and 10 ng doxycycline/ml treated cell lines. The most parsimonious explanation is that there was a mechanical anomaly with the imaging equipment leading to false positive levels of photon acquisition counts over an area of the 24 well plate that contained both the negative control and 10 ng doxycycline/ml treated cells, as these were spatially adjacent during imaging. The 100 ng doxycycline/ml treated cells remained distal from this section of the plate, and therefore may not have been affected by the anomaly, explaining why there was not a corresponding increase in measured bioluminescent flux for all three treatment levels at the 5 h time point.

Levels of bioluminescent flux from cells treated with 100 ng doxycycline/ml were smaller than those detected from similar numbers of constitutively bioluminescent cells in culture. When the luxC and luxE genes were continuously expressed under the control of the EF1-α promoter the maximum level of radiance was measured at 4.5 (±0.16)×10⁵p/s, whereas, with the luxC and luxE genes placed under the control of the tetracycline responsive promoter and induced with 100 ng doxycycline/ml, the maximum measured flux was 1.3 (±0.03)×10⁴ p/s. This is most likely due to a combination of the improved efficiency of the EF1-α promoter as compared to the tetracycline responsive promoter and the increases in transcriptional efficiency imparted during continuous expression. Due to the discrepancy in luminescent flux between tetracycline responsive cell lines and those displaying constitutive expression, increases in treatment levels above 100 ng doxycycline/ml were attempted, however, none were shown to further increase bioluminescent output. These results indicate that the tetracycline responsive lux reporter cells have a relatively narrow detection range, and therefore would not make efficient laboratory reporter strains at this time. There are, however, further avenues that could be explored to improve their performance. The first steps in this direction would be the redesign of the plasmid vectors to regulate expression of only a single lux gene, or the choice of alternative lux genes as points of regulation for reporter function.

Due in part to the poor performance of lux expressing cells to act as a bioreporter for specific compound detection, it was further investigated whether or not constitutively bioluminescent mammalian cells could function as biosensors for toxicological screening. These unique cells are ideal platforms for real-time monitoring of the mammalian bioavailability of potentially toxic compounds, an assay that has not been previously available. To determine their effectiveness in this roll, cells were exposed to n-decanal, a cytotoxic aldehyde similar to the product of the bacterial bioluminescent reaction, and the minimum exposure level capable of reducing bioluminescent production was determined. Decanal was chosen for the initial assay because it can serve a three-fold purpose. As a similar product to that of the reaction catalyzed by the actions of the luxCDE genes, there have long been concerns over the potential cytotoxicity of these types of compound when the lux system is expressed in non-native organisms. By using changes in bioluminescence to monitor for the effects of n-decanal on the HEK293 cell line it was possible to determine 1) if small supplements of the compound can increase bioluminescent intensity, 2) at what level the compound becomes toxic to the cell, and ultimately, 3) if a constitutively bioluminescent mammalian cell line can function as a reporter system for toxic chemical exposure. It was hypothesized that slight increases in n-decanal availability would lead to increased bioluminescent output. Previous work had indicated that when additional levels of n-decanal were made available to cell extracts containing the lux proteins, it was possible to increase overall bioluminescent output in vitro. This effect was not observed during in vivo testing (FIG. 13) however, and none of the time points surveyed produced a result whereby a cell population treated with any level of decanal produced significantly greater bioluminescent flux than the untreated control line. The small, aliphatic nature of n-decanal allows it to cross the membrane of Gram negative bacteria, and our data supports the hypothesis that the saturated ten carbon tail also allows the molecule to pass through the lipid bilayer of mammalian cells. Using the newly developed assay it was confirmed that n-decanal treatment is adversely toxic (high levels of the compound will fix cells in a manner similar to formaldehyde) and will become detrimental to cellular health at concentrations required to generate the diffusive force required to cross the membrane. It is possible, however, that there are alternative explanations for the failure of low level n-decanal treatment to increase bioluminescent flux. The cell could be reaching an equilibrium where the additional influx of aldehyde is boosting bioluminescent production levels, but simultaneously negatively effecting cellular metabolism, thereby reducing overall bioluminescent yield. This situation seems unlikely, given the observation that there is no effect over three orders of magnitude of aldehyde concentration. It is more parsimonious that the low levels of aldehyde concentration do not provide enough diffusive force to allow n-decanal to cross into the interior of the cell through the lipid bilayer. Despite the inability of low levels of aldehyde treatment to stimulate bioluminescent production, it was clear that at and above concentrations of 0.01% the aldehyde became toxic to the HEK293 cell line (Table 8). While treatment with 0.1% decanal reduced bioluminescent output at all time points surveyed, treatment with 0.01% was not able to consistently reduce bioluminescence until 4 h after addition. These results are comparable with previously published reports that demonstrated the ability of aldehyde to diffuse into Gram negative bacteria at concentrations in the range of ˜0.25-50×10⁻⁵ M. Our 0.01% decanal treatment corresponds to a concentration of ˜64×10⁻⁵ M. While the previous experiments used the slightly longer dodecanal in place of decanal (C₁₂ compared to C₁₀), less of that aldehyde is required to enter the cell in order to elicit a similar bioluminescent response because the longer chain aldehydes have been shown to produce a greater bioluminescent signal upon utilization in the lux reaction despite their slower penetration of the cell wall. These data suggest that the concentration range of 10⁻⁵ M is the point where decanal is able to cross the cell wall at a rate greater than it is able to be cleared by aldehyde metabolizing enzymes. The initial production of bioluminescence within error of the positive control indicates that for the first 0.5 h, a sufficient concentration of aldehyde has not entered into the cells to elicit a change in metabolism or cellular health. The fluctuations in bioluminescent production over the next 2 h indicate that aldehyde has entered into the cell, but is likely being processed by endogenous aldehyde metabolizing proteins, whereas the distinct reduction in bioluminescence following the 4 h time point suggests that the concentration of aldehyde has become too great to be cleared and has begun causing cellular damage. The clear distinction between concentrations of aldehyde that affect bioluminescent production, and those that do not have an affect, suggest that constitutively bioluminescent mammalian cells can be used as sensors for monitoring the bioavailability of toxic compounds in real time. Specifically the treatment of cells with 0.01% decanal shows that the real-time nature of the lux expression system can allow researchers to determine not only the presence or absence of an affect from their treatment of interest, but can also do so in a time dependent manner: The autonomous nature of this reaction demonstrated by these results will allow for the development of biosentinel devices capable of acting remotely to detect and report directly on the mammalian bioavailability of a variety of biomedically relevant compounds in a way that is not feasible using substrate dependent luciferase systems.

### Example 4

### Initial Optimization of the Mammalian-Adapted Bacterial Bioluminescence System

The use of mammalian-adapted bacterial luciferase (lux) genes as a reporter system in human cells is still in its infancy. While the initial results detailed in this work are encouraging, the future of the mammalian lux system is still being written. As the newest of the mammalian-compatible reporter options, the lux system has not had the advantage of optimization that comes from widespread adoption and evaluation by multiple research groups. Thus far, all of the popular genetic-based (i.e. those derived directly from living organisms) fluorescent and bioluminescent reporter systems currently being employed for small animal imaging have had the advantage of multiple refinements in order to increase their efficiency under standard laboratory conditions. Perhaps the best example of these incremental improvements has been with the widely used green fluorescent protein (GFP). Originally detailed in 1962, the GFP protein has undergone extensive modification from its native state in the last ˜50 years in order to compensate for the traits that make it less attractive for use as an imaging target. The genetic structure of the GFP protein has been altered repeatedly in order to allow it to fold properly in mammalian cells at the relatively increased temperature of 37° C., to prevent dimerization under the high levels of constitutive expression that are preferred for facile image acquisition, and mutated myriad times in order to alter the signal emission wavelength so that it can be used in tandem with other reporters, or detected with greater efficiency through living tissue. Each of these incremental changes have led to the development of a protein that, while the same in name, in some implementations, cannot even be spectrally identified as its native precursor. The same can be said for alternative bioluminescent proteins.

If use of the lux reporter system spreads to even moderate levels among those actively engaged in optical imaging, there will no doubt be great interest in enhancing its bioluminescent characteristics, just as there has been with other widely adopted reporter systems. To this end, the groundwork for future development and optimization of the lux system has been laid out, with increasing the bioluminescent flux of the system as the primary goal. Under its current implementation, the mammalian-adapted lux system cannot produce levels of bioluminescent flux as high as any of the commercially available bioluminescent proteins can, following amendment with their luciferin substrates. Enhancing the bioluminescent flux of the lux system to achieve levels of flux on par with the alternative systems would overcome this deficiency, which is viewed as the main hurdle to its widespread use in the optical imaging community, and prevent it from being used solely as a niche-based reporter for experimental designs that require bioluminescent production without substrate amendment. To determine points for future optimization of the lux system that could increase bioluminescent production, the function of the lux genes within the HEK293 mammalian cell line was first investigated. While the function of the luxA and luxB genes has previously been evaluated extensively, the focus of these experiments was to evaluate the function of the remaining luxC, luxD, luxE, and frp genes that are responsible for establishment and regeneration of the aldehyde and FMNH₂ substrates required for bioluminescent production. The luxC, luxD, and luxE genes produce protein products that work together in a complex to supply the myristyl aldehyde substrate. Their codependence allows them to be evaluated simultaneously, because a deficiency in any one will adversely effect the production of bioluminescence in vivo. The frp gene acts independently, and therefore was evaluated separately in order to determine its function in the regeneration of cytosolically available FMNH₂. Because the function of these genes is in part dependent on the efficiency of their expression in the mammalian cellular environment, the translational efficiency imparted by the internal ribosomal entry site (IRES) elements that were included to spur translation of the lux genes while mimicking the polycistronic nature of the original bacterial operon was also investigated. The use of a 2A linker site as an alternative to the IRES element was investigated and the resulting changes in in vitro bioluminescent production levels were compared. The 2A element was chosen because it performs the same basic function of the IRES element by generating multiple protein products from a single mRNA under the control of a single promoter element. However, the means by which the protein products are created are very different. IRES elements are relatively large sequences of DNA that, upon transcription into mRNA, form a secondary structure capable of attracting and binding a ribosome to initiate translation of the downstream gene. On the other hand, 2A elements are short, in-frame, linker regions that separate two in-frame ORF's driven off of a single promoter. During translation of the 2A sequence region the nascent amino acid sequence interacts with the exit tunnel of the ribosome, causing a “skipping” of the last peptide bond at the C terminus of the 2A sequence. Despite this missing bond, the ribosome is able to continue translation, creating a second, independent protein product. The short nature of the sequence (they average 10 amino acids in length) and highly efficient 1:1 stoichiometry of these sequences give them many advantages over the more bulky IRES elements. By determining if increased efficiency of the aldehyde and/or FMNH₂ regulating genes increased bioluminescent output in the mammalian-adapted lux system, it allows future research to focus on improving the specific aspects of the lux system that can lead to the most beneficial improvements in the shortest amount of time. Likewise, the comparison of IRES-based polycistronic expression with a 2A-based expression system highlights if the exchange of these linker regions provides tangible advantages beyond the simple reduction in overall system size and repetition of large sequences of DNA in the plasmid vectors. It was not expected that these initial investigations would lead directly to the development of improved lux function in the mammalian cellular background, but instead, that they would provide the framework for moving forward with the first steps of what will hopefully one day be a rich history of lux development.

### Materials and Methods

Replacement of IRES Elements with 2A Elements

Synthesis of 2A Elements:

To determine if the IRES element linking together the luxA and luxB genes in the original pLuxAB construct was detrimental to transcriptional/translational efficiency, it was replaced with a synthetic 2A element. This sequence was previously characterized by Ibrahimi et. al (Ibrahimi, Velde et al. 2009), and was flanked by two identical sequences composed of three glycines, a serine, and three more glycines. The final construct was synthetically assembled and placed upstream of the luxB gene commercially (GeneArt). The purchased construct was cloned into pLux_{AB} using the upstream EcoNI and downstream SalI restriction sites to replace the IRES element, creating the pTa2AluxAB plasmid, which contained a CMV promoter, the luxA gene, the Ta2A linker region, and the luxB gene.

Transfection of HEK293 Cells:

Transfection was carried out in six-well Falcon tissue culture plates (Thermo-Fisher). The day prior to transfection, HEK293 cells were passaged into each well at a concentration of approximately 1×10⁵ cells/well and grown to 90-95% confluence in complete medium. The previously described pLux_{CDEfrp:CO}/pLux_{AB} or pLux_{CDEfrp:WT}/pLux_{AB} vectors as well as the pTa2AluxAB plasmid were purified from a 100 ml overnight culture of E. coli using the Wizard Purefection plasmid purification system (Promega). On the day of transfection, cell medium was removed and replaced and vector DNA was introduced using Lipofectamine 2000 (Invitrogen). Twenty four h post-transfection, the medium was removed and replaced with complete medium supplemented with the appropriate antibiotic. Selection of successfully transfected clones was performed by refreshing selective medium every 4-5 d until all untransfected cells had died. At this time, colonies of transfected cells were removed by scraping, transferred to individual 25 cm² cell culture flasks, and grown in complete medium supplemented with the appropriate antibiotics.

In Vitro Bioluminescent Measurement;

Total protein was extracted from co-transfected pLux_{CDEfrp:CO}/pLux_{AB} or pLux_{CDEfrp:WT}/pLux_{AB} cell lines or the pTa2AluxAB transfected cell line using a freeze/thaw procedure. Cells were first grown to confluence in 75 cm₂ tissue culture flasks (Corning), then mechanically detached and resuspended in 10 ml of PBS. Following collection, cells were washed twice in 10 ml volumes of PBS, pelleted and resuspended in 1 ml PBS. These 1 ml aliquots of cells were subjected to three rounds of freezing in liquid nitrogen for 30 sec, followed by thawing in a 37° C. water bath for 3 min. The resulting cell debris was pelleted by centrifugation at 14,000 g for 10 min and the supernatant containing the soluble protein fraction was retained for analysis. Bioluminescence was measured using an FB14 luminometer (Zylux) with a 1 sec integration time. To prepare the sample for in vitro bioluminescent 150 measurement, 400 μA of the isolated protein extract was combined with 500 μA of either oxidoreductase supplemented light assay solution containing 0.1 mM NAD(P)H, 4 μM FMN, 0.2% (w/v) BSA and 1 U of oxidoreductase protein isolated from V. fischeri (Roche), or oxidoreductase deficient light assay solution (distilled water substituted for the 1 U of oxidoreductase protein). Following the initial bioluminescent reading, samples were amended with 0.002% (w/v) n-decanal and the readings were continued to determine if additional aldehyde could increase light output. All bioluminescent signals were normalized to total protein concentration as determined by BCA protein assay (Pierce) and reported as relative light units (RLU)/mg total protein. All sample runs included processing of cell extracts from HEK293 cells stably transfected with pLux_{AB} as a control for light expression upon amendment.

### Results

Supplementation with NAD(P)H:flavin Oxidoreductase Protein:

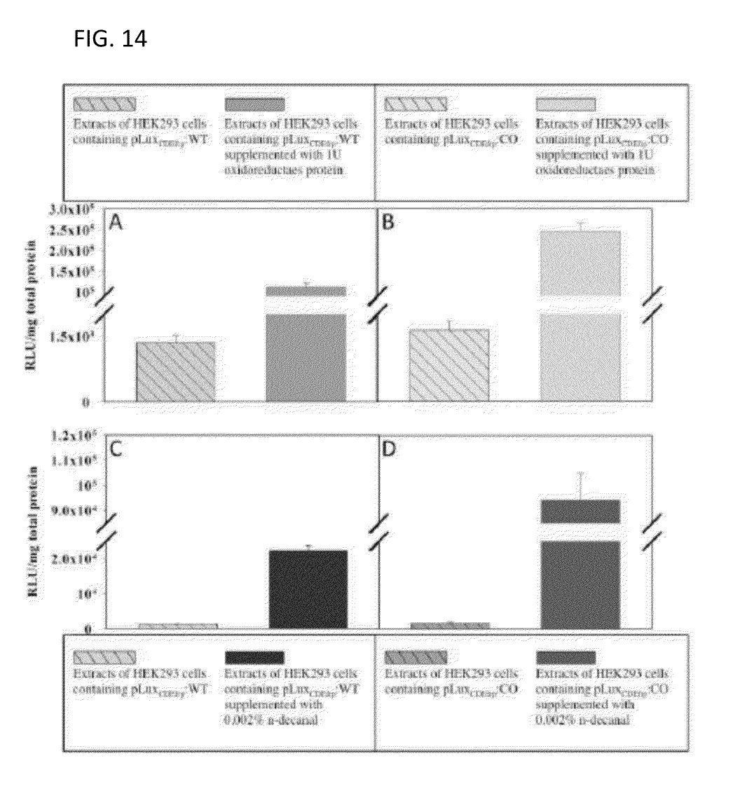

Previous work with the lux system in lower eukaryotes has shown the initial substrate, FMNH₂, to be a limiting reagent in the reaction. To determine if this was the case in HEK293 cells, in vitro supplementation assays were performed with the addition of 1 U of 151 NAD(P)H:Flavin oxidoreductase protein isolated from Photobacterium fischeri. Protein extracts from cells containing the lux genes in either their codon optimized or wild-type forms were subjected to in vitro analysis to determine if the addition of oxidoreductase protein could improve light output. Upon addition of the flavin oxidoreductase protein, the average bioluminescent output increased from 1,400 (±200) RLU/mg total protein to 111,500 (±10,500) RLU/mg total protein in pLux_{CDEfrp:WT} containing cells (FIG. 14A) and from 1,600 (±200) RLU/mg total protein to 245,000 (±20,500) RLU/mg total protein in pLux_{CDEfrp:CO} containing cells (FIG. 14B).

Supplementation with Aldehyde:

The synthesized co-substrate required for light production in the lux system is a long chain aliphatic aldehyde that binds to the luciferase and is oxidized. The ability, conferred by the luxCDE genes, to produce and recycle the aldehyde substrate endogenously makes lux a uniquely beneficial reporter system. To assay for the production of aldehyde, cell extracts were supplemented with 0.002% (w/v) n-decanal, as this has previously been shown capable of functioning in place of the natural aldehyde substrate. When supplied with aldehyde, both the pLux_{CDEfrp:WT} and pLux_{CDEfrp:CO} containing cell extracts showed increases in bioluminescent output. Cell extracts from wild-type

Determination of Bioluminescent Output from HEK293 Cells Containing 2A Linked luxAB Genes:

Five cell lines were recovered that stably expressed the 2A linked luxAB gene sequences following transfection with pTa2AluxAB. These five lines were subjected to in vitro analysis to determine if they were capable of producing more light than approximately equal numbers of cells stably expressing the IRES linked luxAB gene sequences from pLux_{AB}. It was determined that the average bioluminescent signal from cells containing 2A linked lux genes was ˜5500 (±3700) % greater than that from cells with IRES linked lux genes. The major contributing factor to the large standard error of the mean was a single cell line that achieved 20,500% greater bioluminescent production than the pLux_{AB} control (Table 9). When excluded from the calculations, this reduced the average bioluminescent production to ˜1,800 (±230) % over IRES linked gene expression.

### Discussion

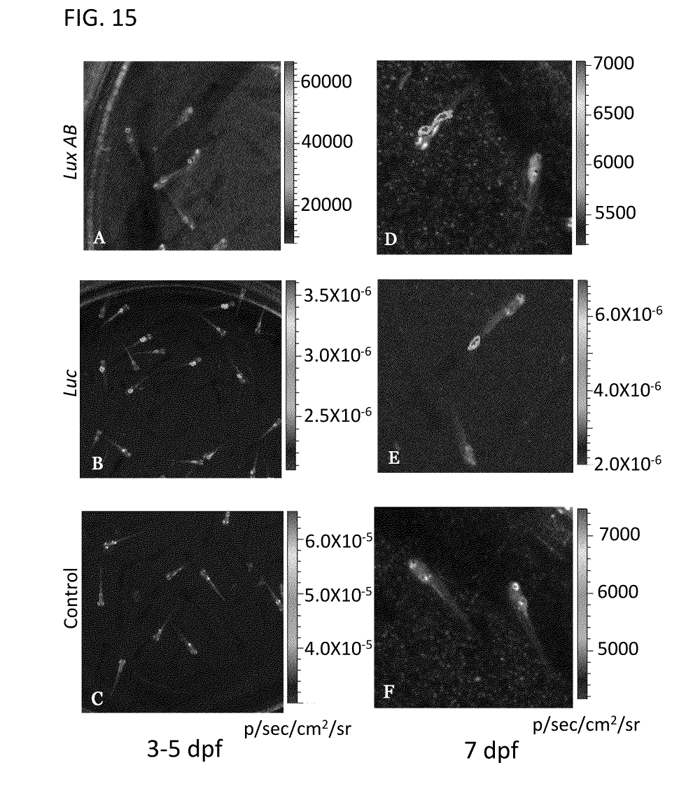

Although the codon-optimized lux system is functioning at a level capable of producing bioluminescent detection under a wide array of conditions, it is clear that concentrations above the available levels of either the FMNH₂ (FIGS. 14 A and C) or aldehyde substrates (FIGS. 14 B and D) will result in increased bioluminescent output. However, an increase in aldehyde production can be cytotoxic, as has been demonstrated in luxAB containing S. cerevisiae and Caenorhabditis elegans cells. This may lead to a scenario where the luxCDE containing cells that most efficiently produce the aldehyde substrate are selected against during the initial period of growth following transfection with luxCDEfrp due to slowed growth and/or elevated cytotoxicity. The increased presence of aldehyde may therefore cause those cells capable of most efficiently producing aldehyde to inhibit their own growth, mimicking the effects of antibiotic selection and causing them to be out-competed in culture by cells expressing lower levels of aldehyde production. Mathematical models of the lux system have indicated that the production of light is much more sensitive to the aldehyde turnover rates modulated by the luxCE genes responsible for encoding the reductase and synthase that convert the myristyl acid to a myristyl aldehyde than it is to the concentration of luciferase dimer formed by the luxAB genes responsible for catalyzing the reaction and facilitating the production of light. The model predicts that a reduction in the concentration of the luxC or luxE gene products will lead to a drastic reduction in light output. If true, then it is hypothesized that the cytotoxicity of aldehyde within the cell may be a non-issue in regards to selecting cell lines that can function in bioluminescent imaging assays. Cells with cytotoxic levels of aldehyde production will be removed early in the selection process due to slow growth rates and inability to compete with faster growing cell lines during the antibiotic selection phase following transfection. Similarly, cells with low levels of luxCDE expression will not generate high levels of bioluminescence during in vitro screening of luxCDEfrp containing cell lines. This would tend to encourage only the selection of cell lineages capable of producing just enough aldehyde to drive the lux reaction, but not enough to impair cellular growth and function, as platforms for biosensor development. Experiments aimed at determining if expression of the lux cassette genes (and, by extension, the products of their associated reactions) altered cellular metabolism and growth rates have supported these predictions. The availability of FMNH₂ appears to contribute as a limiting reagent for the lux reaction in a mammalian cell environment. Supplementation with as little as 1 U of oxidoreductase protein in vitro led to relatively large (up to 151-fold) increases in bioluminescent output levels, while supplementation with 0.002% n-decanal produced less substantial (up to 58-fold) increases in light production. When supplemented with additional oxidoreductase protein to drive the turnover of FMN to FMNH₂, the average production of light increased by 82-fold in wild-type cell extracts (FIG. 14A) and by 151-fold in extracts from cells containing codon-optimized lux genes (FIG. 15B). The increases in light production attributed to additional FMNH2 were consistently of greater magnitude than those associated with aldehyde supplementation. The highest increase in light output achieved through addition of n-decanal was 58-fold in cells containing codon-optimized genes (FIG. 14D), compared with only a 16-fold increase in light output from cell extracts cotransfected with the wild-type genes (FIG. 14C). These results suggest that codon optimization of the remaining IuxCDE genes from P. luminescens allows for more efficient processing of the available substrates in the mammalian cell environment, but does not allow for production levels that rival the ideal conditions of in vitro substrate supplementation where the bioluminescent output would be limited only by the efficiency of the LuxAB luciferase dimer. When supplemented with identical levels of aldehyde, cell extracts containing codon optimized luxCDEfrp genes were able to produce over four times as much light as those containing the wild-type genes (FIGS. 14 C and D). A similar result was obtained under oxidoreductase supplementation, with extracts from the codon-optimized cell lines producing over twice as much light as their wild-type counterparts (FIGS. 14 A and B). The data also indicate that the use of IRES elements is a contributing factor for inefficient bioluminescent expression in the mammalian cellular background. As demonstrated in Table 9, exchanging the IRES element for a 2A element lead to increased bioluminescent output in all cell lines that were stably isolated. It is important to note that during the process of Lipofectamine based mammalian cell transfection, it is not possible to effectively control the location of gene insertion into the genome, nor is it possible to regulate the number of integration events that take place. Taken together, these factors can help to explain the large discrepancy in bioluminescent output between clone number 4 and the remaining pTa2AluxAB transfected cell lines. It is conceivable that the resulting increase in bioluminescent production from cell line number 4 is the result of multiple luxAB gene insertions into the parental cell genome. Assuming all of these insertions remain under the control of the constitutively active CMV promoter, this will afford the cell with multiple locations for simultaneous production of LuxAB protein. Because all cell lines were tested in vitro, each was supplied with an identical level of the remaining required substrates for bioluminescent production. Under these circumstances, the cell line expressing the most LuxAB protein would be capable of producing the most light. To compare and contrast the light output data, all readings are normalized to the total soluble protein concentration from each cell line. This method does not allow for determination of the total amount of Lux protein expression or even the ratio of Lux protein to endogenous protein available during the assay. As a result, there is no way to calculate if the increase in bioluminescent production is the result of multiple insertion events during the course of transfection, or if it is the result of increased up regulation of luxAB gene transcription due to their location within the genome. An additional explanation is that the luxAB genes transfected into clone number 4 were inserted into a euchromatic region of the genome as opposed to a more heterochromatic portion, or that they inserted near another strong promoter, which has caused them to be expressed at even higher levels then would be found under the control of the CMV promoter alone. This explanation is less likely, however, because recent work has demonstrated that the CMV promoter is one of the most active promoters known in the HEK293 cellular environment. Regardless of the genetic reasons underlying the heightened bioluminescent production of clone number 4, it is important to note that the remaining 2A containing clones averaged ˜1,800 (±230) % over their IRES linked counterparts. This increase was relatively consistent (±230%), indicating that increases in this range should be routinely achievable when IRES elements are exchanged for 2A elements. Although it is not yet known whether exchanging the IRES elements governing the expression of the remaining lux genes will have similar effects on autonomous bioluminescent expression, it is clear that the use of 2A elements are an attractive alternative due to their smaller size and increased efficiency at driving downstream gene expression.

### Example 5

### Bioengineering Autonomous Bioluminescence (hoc) in Zebrafish

The ability to investigate physiological processes in live vertebrates in real time is a technical challenge that when overcome will dramatically enhance our understanding of organism physiology, disease processes, and progression of responses to environmental toxicants. In the presence of growing environmental health concerns, the ability to detect and monitor sources of DNA damage has become essential to developing pre-emptive healthcare strategies. The proposed research offers an approach for real-time in vivo assessment of changes in gene expression through the development of a bacterial luciferase (lux) vertebrate (zebrafish, Danio rerio) bioreporter capable of responding to specific exogenous stimuli with autonomous bioluminescent light production. The integration of lux-based in vivo imaging technology into the vertebrate zebrafish genome will provide mechanistic insights of biological responses during chemical perturbations that can be extrapolated towards predictive health-risk assessment in humans. This research effort will create a zebrafish bioreporter containing the complete lux operon, the first autobioluminescent vertebrate of its kind, capable of real-time detection of environmental pollutants. Bioreporters containing a complete lux cassette (luxCDABE) endogenously produce the enzyme (luciferase) and the substrate (tetradecanal) required for autonomous light-production (bioluminescence). Resulting zebrafish offer a repetitive, economical, and non-invasive approach to visually characterize the genetic effects of environmental toxins in vivo for predictive health risk inference to humans.

Stable Integration of the Full Complement of Bioluminescence Genes (luxCDABE) into Zebrafish and Validation of Autonomous, Reagentless Expression Under Constitutive Genetic Controls:

In preliminary experiments, efficient integration of the luxAB genes into zebrafish and validation of substrate-dependent bioluminescence emission has been achieved. To achieve substrate-independent expression, the complete lux operon (luxCDABE) is inserted and expressed. A constitutive bioluminescent reporter is generated by optimizing codon usage patterns in each of the lux genes to match that of D. rerio, creating a vector containing the full lux cassette for microinjection into zebrafish embryos. Stable integration of the lux cassette is assessed via bioluminescence emission profiles while its application as an ecotoxicological biosentinel is evaluated via exposure to a waterborne polycyclic aromatic hydrocarbon (PAH) contaminant.

To achieve this objective, we will develop living zebrafish biosentinels by microinjecting codon-optimized luxCDABE cassettes to form zebrafish transgenics for constitutive and inducible in vivo imaging of biological response during chemical perturbations.

Expression of luxAB in Zebrafish:

As disclosed herein the luxCDABE operon was re-optimized and re-synthesized the and, using a bi-cistronic expression strategy, demonstrated for the first time fully autonomous lux driven bioluminescence emission in an HEK293 cell line within a nude mouse model. The cloning strategy involved a pairwise grouping of the luxCDABE genes and an oxidoreductase (frp) gene, with each pair separated by an internal ribosomal entry site (IRES) element to permit transcription of fused mRNA products from a single promoter. The grouped luxA-IRES-luxB gene fusion was placed under the control of the cytomegalovirus immediate early (CMV) promoter within a pIRES (Clontech) vector for synthesis of the luciferase enzyme. The remaining lux and frp genes were inserted into a separate vector for synthesis of the aldehyde substrate that feeds the bioluminescent reaction and permits self-sufficient light emission. Thus, the two vectors together yield autonomous bioluminescence, whereas the luxA-IRES-luxB vector by itself generates light only if an exogenous aldehyde substrate (n-decanal) is added. Recognizing that IRES elements and CMV promoters were functional in zebrafish and that the codon usage patterns between the mammalian optimized lux genes were similar enough to zebrafish based on GENSCAN predictions (genes.mit.edu), it was hypothesized that injection of the luxA-IRES-luxB vector into zebrafish embryos would result in adequate expression of the luciferase enzyme and emission of measureable in vivo bioluminescence in the presence of exogenously added n-decanal. A linearized luxA-IRES-luxB vector was microinjected into the cytoplasm of single-celled zebrafish embryos and larvae (1-12 days post fertilization (dpf)) were monitored for light production in the presence of n-decanal using an IVIS Lumina CCD imager (Caliper Life Sciences) (FIG. 15). For comparison to existing established reporter models, the luc gene was similarly microinjected into separate embryos. With both reporters, bioluminescence was detected 3-5 dpf. luxAB and luc gene expression was limited primarily to the yolk at 3 dpf and migrated to the trunk and tail by 7 dpf. Bioluminescent output from luxAB incorporated zebrafish was 100 to 1000-fold lower than that of luc incorporated zebrafish. A disadvantage of using lux is its lower bioluminescence output as compared to luc, however, the more intense luc signal rapidly degrades as it depletes its exogenously added D-luciferin substrate (within 1 hour in our mouse models). luxCDABE, however, will remain stable since its substrate is being provided continuously by the cell itself. It is expected that lux emission to increase once the operon is optimized for expression in zebrafish. These results demonstrate efficiently microinjection and establishment lux-directed substrate-dependent bioluminescence in zebrafish. The lux gene cassette is further codon optimized for more efficient expression under zebrafish genetic controls and integrated of the complete lux operon into zebrafish to establish substrate-independent bioluminescence emission.

Codon-Optimization of the luxCDABE Genes:

It is well documented that the codon-optimization process is capable of increasing the levels of transcription and translation of exogenous genes within a host organism. As such, prior to zebrafish expression, the luxCDABE genes from the marine organism Vibrio harveyi is codon-optimized to remove potentially influential regulatory or restriction enzyme sites and to more closely match the codon preference and GC content of D. rerio. The lux genes from V. harveyi are utilized since this is the most well characterized of the bioluminescent bacterial species and its growth optimum of 30° C. most closely matches that of D. rerio (28.5° C.). Using the available gene sequences from GenBank, the V. harveyi luxCDABE genes are synthesized using codons optimized to match the most frequently utilized codon for each amino acid as determined by comparison with all known D. rerio protein sequences.

Preparation of Plasmid Vectors for Integration into the Zebrafish Genome:

Prior to introduction into the zebrafish genome, the lux genes are paired with promoters to drive their constitutive expression and promote increased levels of bioluminescent production following integration. Previous studies have suggested that two of the most active widely available promoters are the CMV and human elongation factor 1 alpha (EF1α) promoters. An efficient vector for expression of multiple genes using these promoters is the commercially available pBudCE4.1 vector. Using this vector as a scaffold, the luxAB genes are placed under the control of the CMV promoter, while the luxCDE genes are placed under the control of the EF1α promoter. This allows for expression of the full lux cassette using only a single vector and streamlines the integration process over conventional two vector approaches. To drive production of multiple gene sequences from a single promoter (luxA and luxB from CMV and luxC, luxD, and luxE from EF1α), the open reading frame of each gene is separated by a 2A sequence to promote the expression of individual protein products from a single mRNA transcript. To promote efficient and stable integration of the DNA construct into the zebrafish genome, the ends of the engineered lux cassette are flanked by I-SceI meganuclease restriction sites commonly used to increase integration and downstream expression of foreign genes in stably transformed zebrafish hosts. The linearized vector are microinjected into the cytoplasm of single-celled zebrafish embryos and larvae screened for bioluminescence in the IVIS Lumina CCD camera.

Determine and Evaluate Bioluminescence Production Profiles Across the Zebrafish Lifecycle:

While the bioluminescence profile characteristics of the lux cassette genes are known under cell culture conditions, they have yet to be elucidated from an endogenously bioluminescent organism. To determine how bioluminescent expression changes over the stages of zebrafish development, bioluminescence is monitored from groups of zebrafish bred from those producing the greatest total bioluminescent flux as determined over the full course of their lifespan. This will concurrently allow for the determination of the effects of lux gene expression on the rate and completeness of maturity during each stage of life.

Selection of Stable Bioluminescent Zebrafish Lines:

Fish expressing optimal bioluminescence serve as a founding (Fo) population, which will be outcrossed to wild type (WT) fish to develop a series of transgenic zebrafish lines (F1). Fish expressing the greatest response are selected to establish a stably integrated (F2) transgenic zebrafish line and a maintainable stock of bioluminescent zebrafish.

Determining Bioluminescent Emission Dynamics Throughout the Zebrafish Lifespan:

Using individuals bred from the stably transformed zebrafish line, triplicate groups are surveyed for bioluminescence production potential at major milestones during their development. Chosen individuals are removed from their housing tanks and transferred to 10 cm petri dishes filled with fish water. When required, anesthetization is performed by treatment with 50 mg MS222/l. Bioluminescence emission is measured with the IVIS Lumina at an integration time of 10 min. Following bioluminescence measurement, fish water containing MS222 are removed and replaced with fresh fish water and the individuals returned to their housing tanks until further testing is required. To fully represent the major developmental stages of the zebrafish life cycle, bioluminescent measurements is taken 1, 2, 10, 35, 50, 90, and 120 dpf. Bioluminescence is determined using the IVIS software to draw regions of interest of identical size over each individual and determining the total flux normalized to photons/second. Bioluminescence from all individuals will then be averaged and recorded for comparison to measurements taken at other stages of development. Of particular interest will be the lowest possible developmental stage where bioluminescence can be reliably differentiated from background, the correlation of bioluminescent flux to zebrafish size, and the stability of bioluminescent flux across the various developmental stages.

Investigating the Effects of Lux Gene Expression on Zebrafish Development:

In parallel with efforts to determine the bioluminescent production dynamics throughout the zebrafish lifespan, it is necessary to determine if that expression is adversely affecting the natural progression of development. Although it has been demonstrated that expression of these genes in other eukaryotic cells does not adversely affect growth, this has not yet been confirmed in the zebrafish model system. To determine if the expression of lux genes alters normal zebrafish development, triplicate groups of individuals maintaining a wild-type genotype are processed in parallel with the groups being surveyed for bioluminescent output. At each of the time points at which bioluminescence is surveyed, both groups are measured to determine average size, and visually inspected to subjectively determine the presence or absence of expected morphological characteristics. All results will be recorded and average sizes of the two groups are compared at each time point using Student's t tests with a cutoff value of p=0.05.

Detail the Use of Constitutively Bioluminescent Zebrafish as Biosentinels for Water Quality Monitoring:

The ability of zebrafish to absorb chemicals directly from water permits their use as environmental sentinels if the chemical/organismal interaction can be observed, i.e., via bioluminescence. A decrease in bioluminescence from a constitutively bioluminescent zebrafish serves as an indicator of reduced metabolism or impaired cellular health potentially linked to water toxicity.

Treatment of Constitutively Bioluminescent Zebrafish with a Simulated Environmental Contaminant:

Zebrafish bred from stably transformed bioluminescent lines are divided into triplicate groupings. For testing, each group is removed from its housing tank and transferred to a 10 cm petri dish filled with fish water. Prior to contaminant addition, the individuals are surveyed for baseline bioluminescent production. Following determination of the baseline bioluminescent level, the dish is spiked with concentrations of the contaminant PAH benzo[a]pyrene ranging from 30 μM to 2 μM, as this has previously been demonstrated to elicit a cytotoxic effect in zebrafish. Concentrations are verified using standard GCMS methods. At time points of 0, 0.5, 1, 5, 16, and 24 hours post contaminant addition the individuals are surveyed to determine bioluminescent production levels. When required, anesthetization is performed by treatment with 50 mg MS222/l. Bioluminescent production is measured using the IVIS Lumina at an integration time of 10 min by drawing regions of interest of identical size over all individuals and averaging the total flux of all individuals normalized to photons/second. Along with all groups tested a group of ten individuals are tested that have not been exposed to benzo[a]pyrene but only to the vehicle control solution used for storage of benzo[a]pyrene to control for changes in bioluminescent production that may occur unrelated to the toxicant treatment. Following bioluminescent measurement, fish water containing MS222 is removed and replaced with fresh fish water, and the individuals are returned to their housing tanks until further testing is required.

Correlation of Contaminant Level with Decreases in Bioluminescent Production:

Using data obtained from bioluminescent interrogation following treatment with benzo[a]pyrene, the change in average total flux from the tested individuals is evaluated to determine the lowest treatment level of benzo[a]pyrene capable of decreasing bioluminescence as compared to the control group using a p value cutoff of 0.05. The overall time required to achieve a decrease in bioluminescence is determined for each toxicant concentration by comparing the untreated control group with the treated group at each of the time points surveyed and determining if there is significantly lower bioluminescent output from the treated group using Student's t tests (p=0.05). EC₅₀ dose response curves will be generated and the magnitude of the bioluminescent decrease at each toxicant concentration and each time point are summarily evaluated.

Development of a Targeted Bioluminescent Zebrafish Transgenic for Autonomous In Vivo Ecotoxicological Monitoring, Using Metal Homeostasis as a Demonstrable Model:

Cloning strategies as detailed above are used to construct a genetic vector with lux-based bioluminescence under the control of the inducible metallothionein promoter to create a living zebrafish transgenic targeted to specific waterborne heavy metal contaminants. Metallothioneins are small intracellular proteins with high affinity for divalent heavy metals. The metallothionein gene (zMT) promoter has been isolated, characterized, and used in zebrafish to drive luc and GFP expression. This fortuitously provides a well characterized and ecotoxicologically relevant model as a comparative platform for our lux-based transgenic expression strategy.

Integration of the zMT Promoter into the luxCDABE Vector and Microinjection into Zebrafish:

The zMT promoter is PCR amplified with appropriate restriction site termini for directional insertion into the luxCDABE vector. It will replace the EF1α promoter to drive inducible expression of luxCDE. luxAB will remain under constitutive control of the CMV promoter. Metabolic modeling of the lux operon predicts this as the optimum control configuration for establishing the fastest and most efficient bioreporter response. The completed vector is microinjected into zebrafish embryos in a linearized conformation with appropriate I-SceI termini. Stable integrates are screened via bioluminescence emission from fish incubated in fish water containing the heavy metal cadmium (25 μM CdCl₂) under the IVIS Lumina imager.

Assess Bioluminescence Production Profiles Across the Zebrafish Lifecycle:

Fish expressing targeted bioluminescence serve as an Fo population and outcrossed to WT fish to develop a series of responsive F1 zebrafish lines. Fish expressing the greatest response are selected to establish a stably integrated F2 transgenic zebrafish line. Kinetics of the bioluminescent response will be evaluated throughout ontogeny (1, 2, 10, 30, 90, 120 dpf) as described. Fish will be exposed to either 25 μM CdCl₂ or standard fish water and evaluated for light production. Developmental parameters are also Assessed.

Establish Response Kinetics Under a Range of Cadmium and Other Heavy Metal Concentration Profiles:

Bioluminescent flux from established zebrafish lines is correlated in a dose-dependent manner to environmentally relevant (pM to μM) ranges of cadmium and other known xenobiotic heavy metal inducers of zMT (i.e., mercury (strong inducer) and arsenic (weak inducer)), using methods described above. Metals shown to not induce zMT will also be used to establish specificity (i.e., Ni²⁺, Pb²⁺, Co²⁺). Mixtures of metals will be tested to establish potential interference problems. The constitutive zebrafish line is used as a control to indicate upper toxic limits within our dose exposure ranges.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

We claim:

1. A vertebrate cell, comprising: one or more heterologous autonomous reporter vectors, wherein at least of the one or more heterologous autonomous reporter vectors comprises a nucleic acid sequence that encodes a bacterial luxA and a nucleic acid sequence that encodes a bacterial luxB.

2. The vertebrate cell of claim 1, wherein the bacterial luxA, and the bacterial luxB are from Photorhabdus luminescens.

3. The vertebrate cell of claim 2, wherein the luxA and luxB are codon optimized for expression in the vertebrate cell.

4. The vertebrate cell of claim 1, further comprising a nucleic acid sequence that encodes a bacterial luxC, a nucleic acid sequence that encodes a bacterial luxD, and a nucleic acid sequence that encodes a bacterial luxE.

5. The vertebrate cell of claim 4, The isolated vertebrate cell of claim 1, wherein the bacterial luxC, luxD, and luxE are from Photorhabdus luminescens.

6. The vertebrate cell of claim 5, wherein the luxC, luxD, and luxE are codon optimized for expression in the vertebrate cell.

7. The vertebrate cell of claim 1, further comprising a heterologous bacterial frp nucleotide sequence.

8. The isolated vertebrate cell of claim 7, wherein the bacterial frp are from V. harveyi.

9. The vertebrate cell of claim 7, wherein the frp is codon optimized for expression in the vertebrate cell.

10. The isolated vertebrate cell of claim 1, wherein the isolated vertebrate cell is a mammalian cell.

11. The isolated vertebrate cell of claim 1, wherein the isolated vertebrate cell is a fish cell.

12. The isolated vertebrate cell of claim 11, wherein the isolated vertebrate cell is a zebrafish cell.

13. A transgenic zebrafish comprising the zebrafish cell of claim 12.

14. The transgenic zebrafish of claim 13, wherein the biosensor detects toxic chemicals.

15. A method of measuring contaminants in water comprising: exposing the transgenic zebrafish of claim 13 to a water sample to be tested, for a time sufficient to allow contaminants to become bioconcentrated within the transgenic organism; detecting the expression of at least one reporter gene; and correlating the detected expression of the transgenic zebrafish to a reference standard comprising an aquatic source containing a known contaminant concentration and thereby determining the quantity of contaminants in the water sample.

16. The method of claim 15, wherein the reference standard comprises an aquatic source containing a known contaminant concentration and thereby determining the quantity of contaminants in the water sample.

17. The method of claim 15 wherein the transgenic zebrafish is exposed to a water sample containing a known amount of contaminant.

18. The method according to claim 15 wherein the contaminant to be detected is one or more contaminants selected from the group consisting of polyaromatic hydrocarbons, electrophilic oxidants, heavy metals, endocrines, and retinoids.

19. An autonomous biosensor, comprising the vertebrate cell of claim 1.

20. The autonomous biosensor of claim 19, wherein the biosensor detects toxic chemicals.
